(19) United States
(12) Patent Application Publication
(10) Pub. No.: US 2008/0046277 A1
(43) Pub. Date: Feb. 21, 2008

(54) Living systems from cardboard packaging materials

(76) Inventor: Paul Edward Stamets, Shelton, WA (US)

Correspondence Address: William R. Hyde, 1833 10th Street, Penrose, CO, 81240, US

(21) Appl. No.: 11/899,562

(22) Filed: Sep. 6, 2007

Related U.S. Application Data
(63) Continuation-in-part of application No. 10/614,906, filed on Jul. 7, 2003.
Application No. 10/614,906 is a division of application No. 10/081,562, filed on Feb. 19, 2002, now abandoned.
Application No. 10/081,562 is a continuation-in-part of application No. 09/790,033, filed on Feb. 20, 2001, now abandoned.

(51) Int. Cl. G06Q 30/00; A01G 9/08; C12N 1/00

(52) U.S. Cl. 705/1; 435/254.1; 47/1.01 R

(57) ABSTRACT

Compositions, methods and business applications of using new and recycled cardboard infused with a plurality of saprophytic (including endophytic) and mycorrhizal fungi matched with seeds of plants (including trees, vegetables, herbs and grasses) whereby the cardboard can be sprouted by end-users to start ecosystems. Such containers may have carbon-credit value for companies and consumers when planted and grown as a carbon sink or carbon offset for the photosynthetic and mycelial sequestration of carbon dioxide. The relative weight of the Life Box's added seeds and spores does not significantly affect the total weight of the infused cardboard, thus not increasing transportation costs.

### BACKGROUND OF THE INVENTION

1. Field of the Invention

The present invention is generally related to business methods, processes and compositions for planting of seeds with simultaneous inoculation with beneficial fungi, new uses of cardboard products, and ecologically sound methods for removing carbon dioxide from the atmosphere to slow global warming while generating carbon credits. More particularly, the present invention infuses cardboard used for shipping containers and boxes with selections of seeds and beneficial fungi that germinate, flourish, and sequester carbon when water and soil is added after delivery.

2. Description of the Related Art

The invention of climate science and the discovery that carbon dioxide traps heat in the atmosphere were among the many scientific accomplishments during the 19th century. In the late 1960's came the realization that the relatively long-lived “greenhouse gases” such as carbon dioxide (CO₂), methane, nitrous oxide, tropospheric ozone and various halogenated compounds and short-lived greenhouse gases such as water vapor could increase average global temperatures, resulting in “global warming” and adverse climate changes. The 1980's saw the first development of climate models and computers that could be used to attempt to quantify the “greenhouse effect.” Much remains unknown, but there is increasingly stronger evidence that human alteration of the chemical composition of the atmosphere will result in various negative, and perhaps even catastrophic, effects.

Although there is some disagreement as to how the earth's climate will respond to the undisputed heat-trapping properties of greenhouse gases, and some uncertainty about other factors both natural and human such as natural climatic variations, the balance between plant respiration and the decomposition of organic matter and photosynthesis, the cooling effects of pollutant aerosols, changes in the sun's energy and ocean absorption and effects, there is increasingly widespread agreement that it wise to attempt to slow the manmade emissions of carbon dioxide and to attempt to increase the removal of greenhouse gases with aggressive and immediate action given the potential severity and threats of global warming and climate change due to human activities. Because of the threat of climate instability and recent record-breaking warm years, and the possibility that human activity may wreak lasting and perhaps irreversible change on the natural world, governments, private organizations and individuals are increasingly looking for ways to deal with emissions of greenhouse gases and the resultant global warming.

The potential human-induced risks and impacts of global warming and climate change include increasing length of warm seasons, threats to water supplies, dramatic drought with loss of soil moisture, precipitation cycles with more frequent and severe storms, heat waves, damage to forests, vegetation and agriculture including loss of fertility and diminished crop yields, increased desertification, the spread of insect-borne and tropical diseases, rising sea levels and storm surges with resultant threats to coastlines and coastal properties, massive extinction of species and loss of biodiversity with disruption of ecosystems, receding of glaciers, loss of snow cover, Arctic ice and Antarctic ice shelves, thawing of the permafrost with resultant release of methane, a very potent greenhouse gas, into the atmosphere, changes in ocean chemistry and loss of coral reefs with resultant effects on sea life, and even possible threats to national security from wars over water, increased instability resulting from rising sea levels and global warming refugees and the resulting chaos that can incubate civil strife.

Changes in the atmospheric concentration of greenhouse gases alters the balance of energy transfers between the sun and the earth by altering the energy balance between the atmosphere and space, land and the oceans. Radiative forcing is a gauge of these changes, a measure of the influence a factor has in altering the equilibrium of incoming and outgoing energy in the earth-atmosphere system. Increases in greenhouse gas concentrations in the atmosphere produce positive radiative forcing, a net increase in the absorption of energy by the earth. This greenhouse effect arises from the greenhouse gases being generally transparent to solar radiation but opaque to long wave radiation, as is greenhouse glass, resulting in a trapping of the absorbed radiative heat of the sun and a warming of the earth's surface.

Carbon dioxide has received particular attention because it is a potent greenhouse gas, because CO2 emissions make-up more than 80% of all greenhouse gas emissions in the U.S. and because atmospheric levels of carbon dioxide are increasing due to “anthropogenic” activities—greenhouse gas emissions and removals that are a direct result of human activities or a result of natural processes affected by human activities. Carbon dioxide is continuously added to and removed from the atmosphere by natural processes; anthropogenic activities, however, can cause additional quantities of carbon dioxide to be emitted or sequestered, thereby changing the average atmospheric concentration.

Carbon dioxide concentration in parts per million by volume (“PPMV”) averaged about 280 PPMV in the pre-industrial period prior to 1750; the current concentration is approximately 380 PPMV (the highest concentration in more than 650,000 years). Worldwide emissions of CO₂ into the atmosphere are now estimated to exceed 27 billion metric tonnes annually, with only some 14 billion tonnes being absorbed by oceans, forests and other carbon sinks (a tonne is a “metric ton,” equivalent to 1,000 kilograms). The concentration of carbon dioxide in the atmosphere is expected to continue rising at an increasing rate unless action is taken. Anthropogenic activities that increase carbon dioxide emissions into the atmosphere include combustion of fossil fuels such as coal, petroleum products and natural gas (the primary source of anthropogenic carbon dioxide, rapidly releasing carbon that was trapped and sequestered millions of years ago), emissions from manufacture, deforestation, agricultural practices that result in soil degradation and loss and the release of carbon dioxide from the soil into the atmosphere (including slash and burn farming and many modern agricultural practices), and accelerated land clearance including road construction and urban and suburban expansion with the loss of trees, shrubs, vegetation and topsoils.

Freeman Dyson was the first to suggest, in 1976, that excess carbon dioxide from the burning of fossil fuels could be soaked up by planting gigantic areas of trees. However, there have been no major advances in large scale tree planting methods.

Trees are an effective means of biospheric carbon sequestration because they remove carbon dioxide from the atmosphere and transform it into carbohydrates according to the formula:
6CO₂+6H₂O+sunlight->C₆H₁₂O₆+6O₂.
The product glucose carbohydrate is utilized to fuel growth and biochemical processes, stored as starch and used to construct tissues and structural and cell wall components of roots, branches, trunks and leaves, resulting in sequestration of the carbon. Utilization of carbon dioxide far exceeds any carbon dioxide that may be released during respiration. The amount of carbon sequestered by a tree or forest during a given period is therefore the amount of CO₂ absorbed through photosynthesis minus that released by respiration.

Trees vary in the amount of moisture they contain, varying, for example, from approximately 10% for Douglas firs and junipers to 50% for basswood as calculated by the green weight to dry weight ratio. However, all trees are approximately 50% carbon by dry weight, with approximately 50% or slightly more being bound in cellulose with almost all the balance being bound in approximately equal parts of hemicellulose and lignin. Healthy forests may store up to hundreds of tonnes of carbon per hectare. Estimates of the total amount of carbon stored in forests range from hundreds of millions of tonnes to over one billion tonnes of carbon. This amount is necessarily a rough estimate due to measurement difficulties, including uncertainties about the “root:shoot” ratio or the partitioning of carbon between the “root” and the “shoot” (above the ground portion), as roots may account for between 10 and 65 percent of a tree's total biomass. Nevertheless, as carbon dioxide is approximately 12/44 carbon, each tonne of carbon stored in trees and forests represents approximately 3.66 tonnes of carbon dioxide that has been removed from the atmosphere. Healthy soils also sequester a great deal of carbon dioxide in the form of carbonaceous material, up to 7% of that sequestered in the plant growth above the soil.

Various “cap and trade” (emission trading) and/or “carbon credit” market-based systems have been developed by international, governmental and/or private entities, including those under the Kyoto Protocol to reduce global warming. However, “carbon offsets,” or carbon credits for the use of “carbon sinks,” such as forest conservation or reforestation and tree planting activities for the removal and sequestration of carbon dioxide from the atmosphere, are in a more rudimentary stage of development.

The most widely used measure of carbon emissions and/or sequestration is the Carbon Emission Reduction Credit (“CERC”). Each tonne of carbon dioxide not emitted due to emission changes or each tonne of atmospheric carbon sequestered from the atmosphere through reforestation or through incorporation of organic matter into soil earns one carbon credit.

Mechanisms being developed under the Kyoto Protocol include Joint Implementations (“JI”), which allow developed countries or companies from those countries to implement offset projects; the Clean Development Mechanism (“CDM”) grants emission credits for projects located in developing countries. The country or companies receive the carbon credits, which may be used, sold or traded. Various private offset and carbon credit systems are also being developed, including informal carbon credits.

Potential problems and complex issues with offset carbon credits include monitoring, measurement and verification of sequestered carbon, regulatory uncertainty including future requirements and land management practices, a lack of organized markets with transfer, title/ownership and trade documentation, high administrative and transactional costs, and indemnification or insurance costs in the event of no CERCs or insufficient CERCs. These problems have prevented standardized, certified and fully audited offset CERCs from being marketed to date. A number of solutions have been proposed; see, for example, U.S. patent application pub. no. 2006/0184445, 2005/0273358, 2004/0230443 and 2005/0283428.

During the 19th century, a number of concepts were also developed and combined to enable paper and paperboard to be transformed into a corrugated cardboard box, including the invention of corrugated (or pleated) paper, the invention of single-sided (single-face) and later double-sided corrugated board, the inventions of machines for producing large quantities, and finally the invention of machines to produce, cut and fold the corrugated box. Since that time there have been relatively few advances in the corrugated box, primarily in printing and the use of recycled materials in boxes. During the same time period, transportation and transport of goods has evolved from horse and boat to trucks, trains and jets. An advance in business methods, processes and compositions for the packaging and shipping of goods utilizing an improved cardboard box that also addressed planting forests and beneficial plants and fungi to increase carbon dioxide capture and thereby potentially decrease global warming would be a desirable advance in the arts.

Corrugated fiberboard or containerboard, usually called corrugated cardboard by non-specialists and also referred to as such herein, is a paper-based construction material consisting of a “fluted” corrugated sheet (“corrugating medium”) and one or two flat “liners” or linerboards. It is widely used in the manufacture of corrugated boxes and shipping containers. The corrugated medium and linerboard are made of paperboard, a heavy paper-like material usually over ten mils (0.010 inch, or 0.25 mm) thick. Paperboard and cardboard are generic, non-specific, lay terms used to refer to any heavy paper-pulp based board, such as card stock, or to corrugated fiberboard, although cardboard might be any heavy paper-pulp based board. “Paperboard” includes not only the corrugated medium and linerboard of corrugated cardboard, but also the other types of paperboard that are used for folding cartons, packaging, containers and egg cartons. Choice and thickness of corrugated medium and linerboard, flute size and adhesive may be varied to engineer end products with specific properties to withstand the forces of packaging, load carrying, and shipping while still maintaining their shape and matching a wide variety of potential uses.

There are three main types of machinery lines producing corrugated board in the United States. Corrugator plants produce corrugated board and typically are also able to convert the corrugated board into boxes, shipping containers, point-of-purchase displays and other types of packaging. Sheet suppliers combine corrugated board into corrugated “sheets” exclusively for purchase by sheet plants. Sheet plants purchase corrugated board (called sheets) and convert into boxes, containers, displays and other packaging. Paperboard arrives at the corrugator in large rolls, is heated, moistened and formed into the corrugated “flutes” on geared wheels and joined to the linerboard (kraft, white, colored or preprinted) with adhesive to form “single face” board on a single-facer. Another linerboard is affixed to the other side of the fluted center to form “single wall” or “double face” corrugated cardboard. Double wall (three sheets of linerboard with two mediums in between) and triple wall (four sheets of linerboard with three mediums in between) corrugated board is also produced to improve strength and puncture resistance. The corrugated board is cut and creased, scored, slotted and/or die-cut to provide controlled bending and folding of the board into boxes. The manufacturer's joint may be secured with adhesive, tape, staples or via stitching.

Perhaps the most significant advances in corrugated cardboard containers in recent decades have been in the use of cardboard composed wholly or partially of recycled and/or tree-free fibers. It is estimated that over 75% of corrugated cardboard is recycled, amounting to approximately 25 billion tons and comprising over 50% of all paper recovered in the United States.

The worldwide production of “cardboard” is over 130 billion square meters, equivalent to 321,237,000 square acres, (84 million metric tonnes), over 35 billion square meters of which is in the U.S. Corrugated board container production accounts for more than 127 million square meters, or 30,888 square acres, worldwide.

Corrugated cardboard, including un-waxed corrugated cardboard boxes and brown paper bags, may be recycled together. Paperboard cartons such as cereal boxes, waxed cardboard used for packaging fresh vegetables, and other non-corrugated boxes cannot be recycled as cardboard but may be recycled with mixed paper products. Typically corrugated cardboard is pressed, baled and transported to a hydropulper or repulper where extraneous materials and contaminants are removed via “ragger” chains, towers, screens, cyclones and/or tanks before pouring onto a moving screen where water is drained before the cleaned fiber mat is rolled, sent through drying cylinders and wound onto spools and into individual rolls. In the U.S. over 70% of corrugated cardboard is recycled; a single fiber from a corrugated box can be recycled many times before it is too short for continued use.

The vegetative, long-lived body of a fungus is an extensive network of microscopic threads (known as mycelium, mycelia or mycelial hyphae) which fully permeates soil, logs, or others substrates within which the organism grows. Most ecologists now recognize that soil health is directly related to the presence, abundance and variety of fungal associations. The mycelial component of topsoil within a typical Douglas fir forest in the Pacific Northwest approaches 10% of the total biomass; the threadlike hyphae of fungal mycelia may exceed eight miles of mycelium per cubic inch of soil, or one mile per gram. Healthy ecosystems include a wide variety of fungal associations. For example, mycorrhizal fungi (including many mushroom fungi) form a mutually dependent, beneficial relationship with the roots of host plants, ranging from trees to grasses to agricultural crops. When the mycelia of these fungi form an exterior sheath covering the roots of the plant they are termed ectomycorrhizal; when they invade the interior root cells of host plants they are called endomycorrhizal (also known as vesicular-arbuscular or VA mycorrhizae). Saprophytic or saprobic fungi (wood and organic matter decomposers) are the primary decomposers in nature, working in concert with a succession of microorganisms and plants to break down and recycle organic and inorganic compounds and materials. Saprophytic fungi have also been found to form symbiotic, mutually beneficial relationship with a number of agricultural crops. For example, corn is known to give bigger yields in the presence of straw bales inoculated with Stropharia rugoso annulata as compared to uninoculated straw bales. The no-till method of farming also benefits from the growth of Basidiomycetes including mushrooms, reducing plant stubble into nutrients. Parasitic mushrooms have their own role in a healthy ecosystem, although they can become overly destructive in unhealthy systems. Another broad class of decomposers is the more primitive, non-mushroom forming “fungi imperfecti,” including also molds and yeasts. Some of these mold-like fungi, like Curvularia species, are both saprophytic and endophytic. Endophytic fungi are a special sub-group of saprophytes which scientists have recently discovered. Endophytic fungi are incorporated into the leaves and stems of plants, and confer benefits of disease resistance, particularly resistance against predatory insects. Plants having endophytic fungi as partners live longer, produce more fruits, and when they die, the endophytic fungi, having already taken up residence, have a ready platform for re-sporulation, thus reproduction. Although most endophytic fungi are ascomycetes, the basidiomycetous wood conk, Fomes fomentarius, has now been found to play a role as an endophyte on birch trees. Once the trees die, these wood conks soon form from the internalized mycelium within the tree, and this species benefits from its early association with the tree, being habituated long before other competitor fungi can invade.

Evidence of the premier role of fungi as decomposers can easily be gathered in a walk through a healthy forest—rotting logs that have been infested by fungi. Without the presence of fungi, few if any organisms are able to effectively degrade the complex aromatic polymers cellulose and lignin, the two primary components of woody plants; cellulose, and particularly lignin, the most recalcitrant of substrates in nature, are generally otherwise resistant to microbial attack and decomposition. The fungi, particularly “white rot fungi,” which are adept at decomposing lignin, and “brown rot fungi,” premier decomposers of cellulose, produce a complex suite of enzymes that oxidize the structures completely to water and carbon dioxide via a radical-mediated mechanism.

For these and other reasons there has been great interest in fungi for uses such as introduction of mycorrhizal fungi, bioaugmentation of soils, bioremediation, biological control and production of mushrooms.

Among the methods for delivering fungal spores and hyphal inoculum to soil for various purposes such as bioremediation or agriculture are carriers such as grain, sawdust and wood chip spawn, alginate hydrogels with and without additional nutrient sources, vermiculite and peat optionally saturated with nutrient broths, vermiculite and rice flour or grain flour, straw or other agricultural waste products overgrown with fungal mycelium, pelleted fungal inoculum preparations, etc. Trees, lawns and seedbeds have been inoculated with mycorrhizal species using various tablets or gels prepared from spores or mycelium. Trees may also be inoculated with mycorrhizal mushrooms by dusting the roots of seedlings with spores or mushroom mycelium or by dipping the exposed roots of seedlings into water enriched with the spore mass of the mycorrhizal species. Another method for inoculating mycorrhizae calls for the planting of young seedlings near the root zones of proven mushroom-producing trees, allowing the seedlings to become ‘infected’ with the mycorrhizae of a neighboring tree. After a few years, the new trees are dug up and transplanted. Another method involves broadcasting spore mass onto the root zones of trees.

Such approaches can be labor intensive, expensive, of uncertain success and/or not suited to widespread or large scale use.

It is also known to add various compositions, including fungi, to seeds to assist growth. However, it is not known to the industry, nor yet practiced, to combine beneficial blends of mycorrhizal, saprophytic, endophytic, entomopathogenic, and/or imperfect fungi with cardboard for the purpose of shipping goods and using the cardboard shipping containers to generate living systems. There remains a need for cheaper and more efficacious methods for large scale use of such unique combinations.

U.S. Pat. No. 4,589,225 (1986) to Stensaas a plant fertilization comprising a primary package containing seeds and a secondary package containing a source of soluble phosphorus, mycorrhizal fungi propagules, including both endomycorrhizal and ectomycorrhizal fungi, and seeds. The primary package may be formed by paper product technology to fabricate a corrugated cardboard-type package. There is however, no suggestion to utilize saprophytic fungi, no suggestion to form the primary package into a cardboard box useful for packaging and shipping goods, and no suggestion that a cardboard box may be planted and thereby earn carbon credits. The reference discloses neither the cardboard box and carbon credit business methods of the present invention nor the novel compositions of the present invention.

In view of the foregoing disadvantages inherent in the known types of fungal inoculants, the present invention provides improved inoculating agents and methods of using such agents.

### BRIEF SUMMARY OF THE INVENTION

The present invention provides business methods, processes and compositions for packaging and shipping goods using cardboard boxes infused with a plurality of saprophytic, saprophytic-endophytic, mycorrhizal and entomopathogenic fungi matched with seeds of plants (including trees, shrubs, bushes, fruits, vegetables, cereal crops, herbs and grasses) whereby the cardboard can be sprouted by end-users to start ecosystems. As these cardboard containers sprout with the addition of water and soil, the living systems emerging from the cardboard boxes become a carbon sink or carbon offset via the photosynthetic sequestration of carbon dioxide, the capturing of carbon via fungal mycelial networks, both of which accumulate carbon credits when the trees or plants grow sufficiently mature. A shipping container is thus easily transformed into a garden or forest or meadow nursery and the possibility of earning carbon credits gives companies and/or consumers added value for using this type of box over others.

The cardboard box starts the process of building soil, with the fungi being the “keystone species” that break down the cellulose, hemicellulose and lignin, in the cardboard box, thus reducing its tensile strength, and releasing nutrients that are made available to the plants, as well as complex biological communities including bacteria, other microorganisms, algae, lichens and/or other fungi. The plants also form symbiotic relationships with the mycorrhizal and saprophytic fungi and thus facilitate a cascade of other biological processes that contribute to healthy soils and healthy plant growth. In essence, biological successionism can be directed through the use of a complex plurality of fungal components, using fungi as the keystone organisms leading the way in habitat enhancement or recovery. The greater the carrying capacity of soil, the more healthy plant biomass can be sustained to absorb CO₂.

In view of the disadvantages inherent in the known products and methods for planting seeds with simultaneous fungal inoculation, the present invention provides improved products, processes and business methods for intensive and/or widespread planting of seeds, inoculation of beneficial fungal species and beneficial use of cardboard packaging materials to initiate and nourish micro and macro ecosystems. The present invention provides new products and methods utilizing cardboard, seeds and fungal spore or hyphal compositions useful for shipping goods and sprouting beneficial seeds and fungi including mushrooms, thereby initiating numerous potential secondary benefits of healthy ecosystems.

Preferred fungi include the fleshy basidiomycetous fungi, “fungi perfecti” (including those fungi producing gilled and polypore and other mushrooms) and the “fungi imperfecti” (the simpler, non-mushroom producing fungi including molds and yeasts) and their various forms of mycelium, spores and conidia, including both sexually produced and asexually produced spores and spore variations. Particularly useful are the saprophytic fungi, including endophytic fungi, the mycorrhizal fungi, the entomopathogenic fungi and combinations thereof. Such products and methods further provide reduced costs, ease of application and improved efficiency when compared to known products and processes.

The present invention has been found to achieve these advantages. Still further objects and advantages of this invention will become more apparent from the following detailed description and appended claims. Before explaining the disclosed embodiments of the present invention in detail, it is to be understood that the invention is not limited in its application to the details of the particular products and methods illustrated, since the invention is capable of other embodiments which will be readily apparent to those skilled in the art. Also, the terminology used herein is for the purpose of description and not of limitation.

### DETAILED DESCRIPTION OF THE INVENTION

Although there is still some disagreement as to the long term effects of global warming, given the potentially catastrophic effects, losses and costs, potential solutions that can be easily and economically implemented are both rare and of tremendous potential value. A criticism of reforestation as a potential solution to excess atmospheric carbon dioxide over what the biosphere and geosphere currently absorb has been that it will “take an area the size of Texas” of additional new forest to absorb that amount of CO₂. As worldwide annual production of corrugated cardboard alone is enough to cover the state of Louisiana, it is apparent that cardboard packaging impregnated with seeds and fungi could help grow enough new plants and trees to remediate industrial carbon dioxide production. When the containers of daily life that are typically discarded become the lush garden or forest, multiple cascade benefits can be expected to ensue. The products and methods provide added value in giving the consumer the ability to further green their environment.

The foundation and continuation of life is directly dependent upon healthy habitats. Habitats are increasingly in peril due to the expansion of human enterprises, exacerbating the effects of erosion, and leading to losses in biodiversity and ecological resilience. In the construction of roads, expansion of suburbia and urban centers, trees and shrubs are removed and topsoils are stripped away and soils are compacted. As rains ensue, the forces of erosion further threaten ecological health in removing latent soils and causing sediment accumulation in the lowlands. This severe loss of topsoil tenacity directly results in enormous expenses both societally and environmentally. Certain human enterprises have also resulted in the contamination of widespread areas with toxic wastes and pollutants.

Compositions, methods and business applications of using new and recycled cardboard infused with a plurality of saprophytic (including endophytic) and mycorrhizal fungi matched with seeds of plants (including annuals, perennials, trees, vegetables, herbs and grasses) allow cardboard to be sprouted by end-users to start ecosystems. Such containers may have carbon-credit value for companies and consumers. The relative weight of the Life Box's added seeds and spores does not significantly affect the total weight of the infused cardboard, thus not increasing transportation costs.

The advantage of this invention is that the fungally infused cardboard become a beneficial platform of growth, helping to protect the newly germinating seeds, and allowing easy placement from a cardboard package that is otherwise discarded or recycled into a non-living medium.

Once the goods are removed, soil is added, and moistened, softening the paper covering the seeds and fungi residing in the grooves of the cardboard. The fungi are first triggered into germination, and the resulting cellulases and hemicellulases, as well as other enzymes, soften the cardboard as the mycelium digests the cardboard and in the process creates sugars and nutrients stimulatory to the germination of the tree seeds, and enabling their healthy growth. White rot saprophytic fungi are particularly useful in degrading lignin, and brown rot saprophytic fungi are particularly useful in degrading cellulose. As the seeds germinate, they pierce through the increasingly softened paper, and growing away from gravity, push upwards. Once the seedlings have emerged, depending upon the density of growth, and the type of tree seeds, the end-user can sub-allocate the tree seeds to pots, or directly into the ground, as it appropriate.

Fungi also present novel advantages in sequestration of carbon. The cellular architecture of the fungal mycelial networks is made of carbon-heavy molecules (chitin, carbohydrates and polysaccharides) and hence habitats infused with mycelium using the present invention significantly enhance their value in terms of augmented carbon credits.

In actively restoring devastated habitats using fungally impregnated cardboard and biodegradable materials, the current invention relies on the naturally gas-governing properties of the selected fungal species. Encouraging the growth of mycelium, and selecting the constellation of fungal species target-specific to the toxic or threatened landscapes, enormous amounts of carbon can be sequestered by the exoskeleton of the mycelial network, heavy in carbon-rich molecules such as chitin and polysaccharides, and/or through the protein-rich contents of the internal cell components. Furthermore, the active placement of mycelial mosaics in a habitat additionally sequesters carbon directly external to its cellular architecture through the production of extracellular enzymes which convert cellulose precursor compounds into arabinoxylanes and arabinogalactans. Furthermore, acids are secreted which bind minerals, thus further sequestering carbon into a stable molecular matrix. Mycelial mats of saprophytic and other fungi may cover areas ranging from small plots to thousands of acres. The mushroom mycelial mat is in fact a carbon bank.

Mycelium is composed primarily of carbohydrates and produces many carbon-heavy compounds, all of which contribute to carbon sequestration greater than simply the measurement of tree and root mass. Fungal mycelial benefits can be expected to increase as CO₂ concentrations rise. Research along increasing gradients of atmospheric CO₂ concentration found near CO₂-emitting springs and research in experimental chambers has shown increasing carbon dioxide concentrations to be associated with near-linear increases in root colonization by soil fungi, increase in total length of fungal hyphae, increase of fungally produced proteins such as glomulin and glomalin and increased soil stability. Increasing CO₂ exposure also increased soil organic carbon and total nitrogen contents as well as microbial carbon and nitrogen contents, resulting in increased storage of both carbon and nitrogen. Rillig, M. C., Hernandez, G. Y. and Newton, P. C. D. (2000). Arbuscular mycorrhizae respond to elevated atmospheric CO₂ after long-term exposure: evidence from a CO₂ spring in New Zealand supports the resource balance model. Ecology Letters 3: 475-478; Rillig, M. C., Wright, S. F., Allen, M. F. and Field, C. B. (1999). Rise in carbon dioxide changes soil structure. Nature 400: 628; Ross, D. J., Tate, K. R., Newton, P. C. D., Wilde, R. H. and Clark, H. (2000). Carbon and nitrogen pools and mineralization in a grassland gley soil under elevated carbon dioxide at a natural CO₂ spring. Global Change Biology 6: 779-790. New Phytol. 147: 189-200; Treseder, K. K. and Allen, M. F. (2000). Mycorrhizal fungi have a potential role in soil carbon storage under elevated CO₂ and nitrogen deposition.

The present invention is applicable to all cardboard containers, boxes and products. By way of example, but not of limitation, either corrugated or non-corrugated cardboard material, linerboard, pressed cardboard, paperboard, fiberboard, containerboard, foodboard, boxboard, card stock, paper or any cellulosic, ligninic, biodegradable polymer or paper based membranes may be used for cartons, holders, boxes, overslips, envelopes, postcards, packing peanuts, noodles or shredded material, and retail display cases. Even the shredded cardboard material retains value as a source of living systems, and gains ease of use when, for example, added to compost or mulch and spread by hand or added to hydromulch for large scale dispersion with hydroseeders or spray hydroseeders or other equipment. Boxes and containers incorporating seeds and fungi still serve their traditional, structural function for the delivery of goods, but now have increased value for their after-delivery use. The panels of the box host assortments of seeds customized to the ecological and cultural specifics of their destination.

As consumer products and their shipping boxes account for most of the unrecycled cardboard in this country, such are particularly suited for use with the present methods and compositions. Examples include DVD boxes, CD boxes, shoe boxes, overwrappers, book boxes, frozen food boxes, bulk canned good boxes, bottled water boxes, pet food boxes, egg cartons, pizza boxes (which many recycling centers do not accept because adhering food interferes with machinery and processes), cardboard insulated covers and wrappers for coffee cups, computer equipment boxes, furniture holding boxes, flat stock separators used for the interior linings within boxes, boxes for carrying relief supplies to refugee communities, cardboard separator panels, cardboard cushioning components (including shredded cardboard), etc.

Either the entire cardboard item or a portion thereof may contain the seeds and spores; for example, only one panel of a DVD box may be seeded and spored, or a seeded and spored insert may be included with the box.

The present invention is well suited to plant and tree species (such as Pseudotsuga menziesii, Douglas fir) that require cold shocking or stratification before germination rates peak. Some species may need weeks or months of cold temperatures to achieve peak germination rates. Corrugated cardboard utilizing seeds that had been frozen and, two weeks later, incorporated into panels sprouted three weeks after planting. Frozen food boxes, or boxes such as pizza boxes that have been pre-frozen, confer the additional advantage of stratification for those tree seeds which require cold shocking to enable germination. A pizza box, when filled with moist soil and watered after use, becomes a seed tray for any desired plant and fungal species. Seeds that need fire or heat above 49 C. (120 F.) to germinate, or whose germination is enhanced or activated by heat, including the coastal redwood Sequoia sempervirens, the giant sequoia Sequoiadendron giganteum and the ponderosa pine Pinus ponderosa, may be heated before incorporation into the cardboard, or heated during the manufacture of the product by incorporation into the pulp during manufacture of the cardboard or fiberboard (which typically involves heating or baking during molding, rolling or manufacturing processes). With some types of glues, the seeds may be incorporated into a “melt” at high temperature to aid germination. Such seeds could alternatively be infused into the cardboard overslips holding hot beverages, such as coffee. The heat exposing the cardboard overslips containing these seeds would then activate them, making germination more likely. Spores of heat tolerant mushroom-forming fungi such as Coprinus niveus, Coprinus coprophila, and other Coprinus species may optionally be included.

Another embodiment of the invention utilizes laminating of paper based products, between which mutually compatible fungal spores and seeds are placed during the manufacturing process. Laminating involves the binding of sheets of paper, cardboard, chipboard, and other lignocellulosic membranes using adhesives. The formulation of adhesives can be designed to benefit fungal spore germination. The advantage of using lamination is that spores and seeds can be laid evenly over the surface. An unobvious advantage is that the mycelium emanating from the germinating spores grows or ‘runs’ faster on a planar surface, two dimensionally, than three dimensionally. Such spores, upon germination, are positively influenced by the planar configuration of lamination to produce rhizomophs, fan-like growths that soon coalesce into cellular sheets of mycelium. These sheaths enmesh the seeds and, moreover, produce protective mycelial membranes, thus nurturing the seeds while forestalling or preventing intrusion from parasitic organisms, especially free-falling airborne spores. The laminated surfaces are further knitted together by the infusing mycelial networks. These newly formed fungally bound membranes better retain water, up-channel nutrition, and fully envelop seeds in a plurality of protective membranes originating from saprophytic, saprophytic-endophytic, and mycorrhizal fungi. When the laminated sheets include a fluted corrugating medium, the ensuing mycelium runs faster in the valleys of flutes than over their peaks. Hence when seeds are place in the valleys of the flutes, the mycelia more quickly makes contact.

A major advantage of the present invention is that it adds only a negligible amount of shipping weight as the seeds and spores weigh very little. Valued and desirable tree species such as Pseudotsuga menziesii (Douglas fir), Sequoia sempervirens (coast redwood) and Sequoiadendron giganteum (giant sequoia) have tens of thousands of seeds per pound; Alnus sinuate (Sitka alder), Betula papyrifera (paper birch), Picea sitchensis (Sitka spruce) and Thuja plicata (red cedar) have hundreds of thousand of seeds per pound. A billion spores of saprophytic, endophytic and/or mycorrhizal spores may weigh less than a gram. Thus, for example, a hundred seeds and a million spores may add less than a gram, or depending on the seeds, grams to the shipping weight of the largest and smallest box and having a negligible effect on shipping rates. Boxes can be customized by zip code destination to deliver ecologically appropriate seeds ands fungi. The cost of using seeded and spored cardboard can be justified as an economically valuable, cost-effective product and procedure for incorporating carbon dioxide into fungi and plants in both microsphere and biosphere.

Manufacture of cardboard and fiberboard boxes is an old art, and techniques of incorporating seeds and spores, or carrier materials containing seeds and spores, at various stages of cardboard manufacture will be readily apparent to those skilled in the art. Spores and seeds may be pre-mixed or added separately. Cardboard portions may be pierced or perforated to ease germination and growth of seedlings.

The interior face of corrugated cardboard, such as the interior surface within a box, is preferably of thinner paper than the fiberboard or other stock used on the outside surfaces of the box. For corrugated products, the use of fungally and seed friendly natural glues made of wood, vegetable products, agars derived from seaweeds, and even synthetically manufactured adhesives are anticipated to be most appropriate to different sets of mixes of seeds and spores. Clay type mordants, mixed with other adhesives such as those used currently in affixing paper to corrugated cardboard or sticky tapes may also be usefully employed. Montmorillinate clay is a useful carrier for mycorrhizal spores, containing useful nutrients helpful to both plants and fungi, with the water holding capacity of the clay proving beneficial in maintaining moisture in the panels during activation.

The spores and seeds may be first mixed together and then immersed into a liquefied glue which is used with a thicker lower sheet of corrugated cardboard stock and/or corrugating medium and a thinner sheet of paper as the second face, thus allowing the seeds to more easily penetrate the thinner overlayer upon germination post soaking with water. Such adhesives can be applied in a slurry, facilitating the mixing of spores, seeds, and its application to the manufacturing process. Electrostatically enhanced application technologies are also anticipated as being useful within the scope of this invention.

Traditional wood fiber based cardboards and papers may be utilized, but preferably use is made of recycled and tree-free cardboards and papers such as, for example, those of hemp, grain straws, kenaf, jute, coconut coir, bamboo, switch grass, grasses, cotton, corn, coffee, cellulosic materials, cellophanes (including those with silicon fibers) and biodegradable polymers. Corrugated cardboard and paperboard and related materials are ‘clean’ enough and structurally selectively favor the fungal mycelium so that products constructed of such may be utilized without pasteurization or sterilization.

Materials such as the corrugating medium, liners or paperboard may optionally be amended to provide additional nutrients via spraying or soaking of the materials in sugars such as maltose, glucose, fructose or sucrose, molasses, sorghum, mannitol, sorbitol, corn steep liquor, corn meal and soybean meal, vegetable oils, casein hydrolysate, grain brans, grape pumice, ammonium salts, amino acids, fertilizers, plant growth and germination hormones, enhancers and accelerators, yeast extract, vitamins, etc. and combinations thereof. Typically such amendments should be utilized sparingly or with materials that are to be pasteurized or sterilized, as such amendments, particularly carbohydrates and nitrogen supplements, may greatly reduce substrate semi-selectivity for fungi and increase the risk of contamination after fungal inoculation.

The spores or fungal hyphae transfer agents may optionally contain further amendments including germination enhancers, growth enhancers, sugars, nutritional supplements, surface active and wetting agents, spore and hyphae encapsulating materials, yeasts, bacteria, fungi imperfecti, algae, lichen, etc. Fungal hyphal mass can optionally be dried or freeze-dried and packaged, with or without additional spores, in spoilage-proof containers for marketing to end users as a seed and slurry additive.

Glues include starch-based glues, wood-based resin glues, vegetable-based adhesives and adherents, malt and sugar glues, agar, tapioca, Elmer's®, and slurries of paper fibers, reformed into sheets and imbedded with spores and seeds. Binding agents or “tackifiers” may be employed as a component in addition to the glue utilized to attach the layers of corrugated cardboard. Various binding agents and tackifiers are known to those skilled in the art; see, for example, U.S. Pat. No. 5,459,181 (1995) to West et al.

Detailed instructions for each configuration can be included with or printed directly on the panel holding the seeds and spores, and can also be referred to on an interactive website which the customer can access. This website also can record the serial number of each seed and spore panel, so that, for instance, tree growth and carbon sequestration can be documented through time and space.

Upon unpacking the box's contents, the box is, depending on shape, used intact or disassembled by hand or by sharp instrument. The cardboard panels, infused with seeds and fungi, are laid upon or into soil. With the addition soil (for example, 12-15 mm.) and water, the cardboard softens, the fungi are activated, and the seeds germinate. Immediately upon germination the seeds have contact with beneficial fungi, insuring an early symbiotic relationship before competitor fungi can harm the seeds. The mycorrhizal fungi stimulate shoot and root growth, expand the sphere of the root zone for absorption of water and nutrients, improve the micro-hydrology of the surrounding soil, and protect the young plants from diseases. With moisture, the saprophytic fungi decompose the cardboard, freeing more nutrients. The cardboard layer lessens evaporation, preserves moisture, shades and cools the soil underneath. The softening cardboard allows the penetration of the shoots and roots. If the cardboard is scored, penetrated or pierced with fine cuts during manufacturing, the roots and shoots can emerge less encumbered. The cardboard fully decomposes, becoming soil, and leaves no waste.

Double face corrugated cardboard with one face being a paper overslip has been observed to give superior results as compared to single face sheets with germinating seeds on open corrugated flutes, both the paper and the open corrugations facing up. When moist soil was placed on top, those with a cover paper overgrew with saprophytic fungus and the sprout pushed up through, and more vigorous sprouts emerged as compared with the open corrugations. The panels with visible saprophytic fungi showing on the cardboard were observed to have the best seed germination rate. Paper is typically preferred over a thick fiberboard as for the upper sheet for planting, as fiberboard may inhibit successful germination of smaller seeds.

Life box panels afford the end-user with ease of use, depending upon the delivery system. For instance, the cardboard containers that hold bulk foods and goods as seen in most grocery stores, typically have 2-4 inch side walls, which makes for an ideal seed tray nursery.

Tree seeds are preferably incorporated at a rate of 0.1-100 tree seeds per square inch, more preferably at a rate of 1-10 seeds/sq. in. and most preferably 2-5 seeds/sq. in. Grass, vegetable, herb, etc. seeds may be used at a higher rate. Endomycorrhizal, ectomycorrhizal and saprophytic fungal spores are preferably incorporated at a rate of 1-10,000 spores per square inch, more preferably 100-1,000 per square inch and most preferably at 1,000-10,000 or more per square inch. If mycorrhizal, saprophytic, endophytic or mycopesticidal fungi are used in concert with compatible seeds of plants, the cardboard panels become springboards for life and ecological recovery.

Although the seeds and spores in the package may only contain of few grams of organic substance, with proper nurturing, their downstream growth could accumulate thousands of kilograms of carbon-rich fibers in the forms of lignin, cellulose and hemicellulose as well as carbohydrates. As with any young plant, aiding the young increases its chance of survival and ultimate maturation. Adding saprophytic, endophytic-saprophytic and mycorrhizal fungi aids the young seedlings survival by helping retain moisture, absorb nutrients, enlist other mutually beneficial organisms while staving off disease, competitors and parasites.

One advantage of using a combination of fungi and seeds with cardboard as a platform for growing an ecosystem is that the saprophytic fungal spores, upon germination, produce enzymes which degrade the cellulose and lignin, and as the fibers are degraded, the tensile strength of resistance of enveloping cardboard is likewise degraded, allowing for the seeds to more easily penetrate through the cardboard liners. Additionally, the use of mycorrhizal fungi help the germinating seeds gather nutrients, resist disease, and vitalize them. Moreover, the use of endophytic fungi helps the young plants thrive, as the fungal cells become incorporated within the shoots and leaves, staving off parasites and providing nutrition from metabolic waste products. Adding saprophytic, endophytic-saprophytic and mycorrhizal fungi aids the young seedlings survival by mycelial expansion of root zones, helping retain moisture, absorb nutrients and enlist other mutually beneficial organisms while staving off disease, competitors and parasites. The use of all three fungi—saprophytic, endophytic and mycorrhizal—when combined with seeds of plants, particularly trees, and infused into cardboard creates a unique synergistic platform for beginning the process of carbon sequestration and mitigating global warming.

The use of such cardboard boxes with walls have been contain plant seeds in combination with fungal spores or mycelium of mycorrhizal, symbiotic, saprophytic, and/or entomopathogenic fungi solves a multiplicity of problems with one solution. The prevalence of cardboard boxes delivered throughout the world on a daily basis exceeds thousands of tons per day, boggling the imagination. The cardboard box is ubiquitous to the world community. The predominance of cardboard in the manufacturing of boxes and its over-abundance strains the resources of communities. With this invention, cardboard boxes have a value-added, after market benefit as they become a living resource for ecological recovery. The panels of the box can be used for home gardening, commercial agriculture, for mycofiltration, mycoremediation, and mycopesticidal purposes. The box can be used as an educational tool for teaching children while at the same time be the container for transporting items related or unrelated to the invention. The cardboard boxes become an ecological footprint for creating a garden, seed bed, an orchard, a forest and even an expanding oasis, starting the process of habitat improvement and recovery. An added advantage is that the cardboard panels can be placed over soil to suppress competitive weed growth and to retain moisture. The decomposition of the paper based materials by the fungus releases nutrients to aid, preferentially, selected plant growth.

Nearly all plants have joined with saprophytic and mycorrhizal fungi in symbiosis. Plants may devote a majority of the net energy fixed as sunlight to below ground processes, not only root growth but also to feed mycorrhizal fungi and other microorganisms. However, this symbiotic relationship is not a net energy loss. Mycorrhizal fungi surround and penetrate the roots of grasses, shrubs, trees, crops and other plants, expanding the absorption zone ten- to a hundred-fold, aiding in plants' quest for water, transferring and cycling macro and micro nutrients, increasing soil aeration and the moisture-holding capacity of soils and forestalling blights, pathogens and disease.

Such mycotechnologies also provide means for introduction and companion cultivation of saprophytic mushrooms with agricultural crops. The benefits of mycorrhizal fungi are well known; the present inventor and others have also found that companion cultivation of saprophytes enhances both quantity and quality of yields of grains and vegetables and other crops. As mycelia bind soil particles (aggregation), soil compaction is decreased and aeration is increased, allowing roots, oxygen, carbon dioxide and water to move through the soil. This improvement in soil quality may be noticed as a ‘bounce factor’ when walking over soils inoculated with saprophytic fungi. For example, Hypsizygus ulmarius on sawdust, covered with straw, has been found to be of great benefit to many crops and plants, including corn, beans and Brussels sprouts; large ears of corn were produced in a poor experimental soil, whereas previously the present inventor had not been able to successfully cultivate corn in his garden due to growing season and climate limitations. Hypholoma sublateritium was also of great benefit to corn cultivation. Stropharia rugoso annulata is known to benefit corn and was found to provide such a benefit, particularly in the second and following years after inoculation. Thus companion cultivation of saprophytes also offers preferred methods of improving gardens and crop yield while reducing the need for fertilizers. See Pischl, C., Die Auswirkungen von Pflanzen-Pilzmischkulturen auf den Bodennaehrstoffgehalt und die Ernteertraege (1999), Master's Thesis, Leopold-Franzens-Universitat Innsbruck. Mushrooms were observed fruiting underneath seedlings, the dewdrop formation and drip zone providing a preferred fruiting site. However, the plants and mushroom species must be carefully matched: while the Oyster-like mushroom Hypsizygus ulmarius had a beneficial effect on some neighboring crop plants, the Oyster mushroom Pleurotus ostreatus did not (Pischl, 1999). On the other hand, for nematode infested soils, P. ostreatus and other Pleurotus species may be preferred, the mycotechnologies herein acting as a nematode-control delivery system.

Preferred offset carbon credits include monitoring, measurement and verification of sequestered carbon, regulatory certainty including future requirements and land management practices, organized markets with transfer, title/ownership and trade documentation resulting in CO₂ standardized, certified and fully audited offset CERCs.

Information for application and registration is preferably included as well as information on how to bank and monetize the carbon credits if the trees are planted and nurtured to an advanced stage. A system of accounting basis unit sales allowing donation or sale of the credits is also desirable. Emerging “green tags” and “gold standards” for carbon credits are desirable, as are field verification, accounting requirements, fungibility, and trading systems or exchanges.

As the carbon credit system is still developing, such carbon credits may ultimately take many forms aside from monetary sale or cash redemption, including carbon tax credits, income tax credits, gas tax credits, sales tax credits, reduction of pollution related tariffs and fines or other tax credits or deductions.

Optional uses of the present business methods and compositions with carbon credits include manufacturer's or other credits or rebates arising from use of a box to generate certified CO₂ offsets. If a customer has accumulated 100 tons of carbon credits, a company may take the surplus carbon credits of the customer as payment for goods or services. Alternatively, this form of carbon credit accumulation from the placement and growth of such living system boxes could be used for the purchases of items in the marketplace where the goods that are being sold are from companies which also subscribe to mutually recognized carbon credit exchanges.

The timber company, paperboard manufacturer, corrugated cardboard manufacturer, box manufacturer, product manufacturer, wholesaler, retailer, distributor or box buyer, shipper, delivery company and/or consumer may optionally get a share of any carbon credits. The invention is of particular benefit between commercial entities, particularly the manufacturer of the fungi-plant infused boxes and their purchasers. As an example, companies could purchase quantities of the boxes from a manufacturer, and the offsets of carbon realized from growing trees could benefit both. The boxes could germinate through a company logo, for instance, or along a prescribed ink-printed path, which would correlate to the printed content on the outer liner. Luminescent fungi could be embedded into the container so that the emerging mycelium could glow-in-the-dark, according to a predetermined path, giving rise to an emblem, wording, or other images useful to the consumer.

The business methods, processes and compositions utilizing cardboard, seeds and spores provides the opportunity for the consumer to grow trees and sequester carbon dioxide from the atmosphere. The two developing sequestration processes are interdependent. The inventor anticipates that cardboard infused with seeds, of, for instance, of sequoia trees, will qualify for carbon credits much as trees qualify when they are planted as seedlings and achieve growth independence as they mature. As trees emerge from seeds into seedlings, and eventually into towering adults, tons of carbon are sequestered into their cells. Mycelial networks will provide additional carbon sequestration as a keystone to rich and abundant soils. This sequestration will occur over time with initial values low. Actual carbon sequestration will of course depend on a host of factors such as locale, rainfall, tree species, age and soil type, or die-back due to drought, disease, inadequate nutrition, insect predation, and grazing by herbivores. Young growing trees, shrubs, bushes and grasses will accumulate carbon; irrespective of these factors, a tree that matures to a mass holding one ton of carbon is equivalent to one carbon credit. Eventually forests store more carbon than other types of land use; bare and barren ground have little fungal mycelium and few plants with little carbon sequestration and little capacity for CO₂ absorption.

As long as the wood of their trunks and branches continues to exist, either in living forests or sustainably harvested forests and a host of forest products such as furniture, kitchen utensils, buildings, fences, infrastructure, etc., the wood of the new trees that we plant today will continue to have locked within it untold tons of carbon that would otherwise have remained in the atmosphere.

As carbon credit systems elaborate to match the increased needs and applications, permutations of carbon credits will increasingly reward those starting new floristic ecosystems in places currently anemic in plant growth such as deserts or forest fires. Using living cardboard as a cover over arid soils captures and retains moisture otherwise lost to evaporation. As the fungal spores and seeds germinate, their sequestration of water also enables carbon sequestration as fungal cells proliferate. The fungal exoskeleton of the mycelium is carbon rich, and, external to the mycelial exoskeleton, many fungal species produce oxalic acids, which are characterized by the joining of two carbon dioxide molecules. Furthermore, fungal mycelium produces polysaccharide shields, a mucilaginous, glomulin-like substance, from their emerging cellular tips, hydrating habitats in advance of cellular contact. These three zones—the mycelial cells, the oxalic acids and the resulting calcium oxalates, and the mucilaginous substances are all heavy with carbon. Hence soils that are rich in mycelium, are also rich in fungally associated carbons.

Boxes and containers may be assigned a tracking number such as serial numbers, bar code numbers or other means of identification for tracking over the life of the box and field monitoring and verification for carbon credit purposes. By having the previously described fungi and seed infused cardboard boxes and containers serialized, the manufacturer, vendor and recipient can trace its distribution.

Moreover, each box can refer to an interactive, computer accessed Internet website. This website can be used to provide support materials, gather reports of successes and failures and allow recipients to engage in social communication. Such website will preferably gather and display data from all such boxes and containers, including where they originated, the mixtures of seeds, their planting destinations or location, survival rates, growth or development, and carbon sequestration (carbon mass) results, and thereby create a traceable chain-of-custody over the lifetime of the developing trees (and other plants). Once the customer verifies the location of planting, the planting may optionally and preferably be monitored and verified over time via satellite photography or other means such as aerial or ground photographs. The website becomes a multigenerational hub for teaching and passing knowledge on to students, children, adults, and institutions while having the net effect of verifying the carbon credit accounting. Such an interactive website would also engage the end-user recipient to become part of the solution for global warming and carbon sequestration, and be inspirational to others, particularly children, teachers, educational institutes, corporations, religious organizations, governmental agencies, and NGO's (Non-government organizations).

All such information that may optionally be incorporated into the website or transferred over the internet may also be printed out and mailed or otherwise shipped, in a cardboard/seed/spore wrapper), without the use of computers or the internet. As verification is the primary purpose, a recipient, landowner could do this by mail, paying a satellite service to print images of their emerging forest and have a calculation of reforestation and carbon credits generated.

Web-traffic through a custom designed Internet site could generate many business benefits and opportunities. For instance, the website could list participating companies, and encourage visitors to support these companies with their purchases. The website could offer products such as spray misters, bug traps, humidity domes, trays, books, videos, and could even adopt a multi-level marketing, or sublicensing structure, empowering consumers to become agents for marketing these boxes which emerge into ecosystem. Such boxes and containers could be marketed as “Living Systems” or “Life Boxes” and become a standard proof of carbon sequestration that would be viewable to all, and hence self-adjusting with each customer's input. Such an Internet web-based system of tracking the emergence of ecosystems from cardboard containers having a plurality of beneficial fungi and paired with appropriate seeds, would bring certitude to the carbon credit exchange system, giving verification to the carbon credit economy, which is currently lacking. Such a website would give credence to the promise of carbon sequestration—the website would verify it. Current satellite imaging tools are useful now for authenticating reforestation, and undoubtedly will improve in the next few decades.

Companies which stay in existence, for example, 30 years after delivering the cardboard boxes, can verify the trees exist, and with the increase in satellite observation tools used by such companies as Google® Earth (http://earth.google.com/), the net effect of life box panels can be verified as the trees mature. Such a method of verification separates intention to sequester carbon versus actual sequestering of carbon. Companies adopting this approach can book carbon credit receivables, realizable in whatever duration of years is needed for the tree(s) to amass one ton of carbon. Since the carbon credit system addresses long term effects, this invention addresses issues which otherwise confounds the interpretation of carbon credits and which is at the center of ongoing debates. If a company or government determines an overall success rate from germination to seedling to sapling to maturing adult trees, the knowledge gained will be convertible to tons of carbon as ecological capital, or monetary form. With each year, carbon credits incrementally accrue to any one participating party. If one establishes the number of trees, their age and size, and the number of customers who bought and planted boxes, the data field would reflect the sum of carbon actually sequestered, not a hypothetical carbon credit. Such a system would be self-correcting as the data bases are updated with current information from customer data inputs. Emails requesting or giving updates can be generated annually, and as the trees mature, the data would become more accurate in terms of total carbon actually sequestered. As such the system is automatically self-adjusting and increasingly meaningful in carbon credit worthiness.

As this invention is designed to last many lifetimes, with the trees for instance, surviving to hundreds of years in age, it is expected that satellite imaging tools such as Google® Earth will improve in imaging abilities, thus allowing for verification that trees have been planted, grown, and sequestered carbon. Not only will the carbon be sequestered in the above and below ground physical form of the tree, carbon sequestration from the emerging fungal mycelium will be significant.

Numerous benefits of trees have been pointed out; see, for example, the National Arbor Day Foundation publications and Brack, C. L., Pollution mitigation and carbon sequestration by an urban forest. Environmental Pollution, March 2002, 116 Supplement 1, p. S195-S200. Beyond mitigating global warming and storage and sequestration of carbon, these benefits include pollution mitigation and improvement of air and water quality, reduced noise pollution, amelioration of urban climate extremes and urban heat islands, cleaner air, increased numbers of birds and songbirds, increases in property values, reduction of runoff into rivers and streams, lower temperatures of parked cars, reduced volatilization of bitumen from asphalt, conservation of energy both summer and winter with reduced consumption of electricity for heating and cooling, increased privacy, improved microclimate, outdoor recreation, aesthetics, beautification and quality of life, sustainable food and habitat for wildlife and a source of general and specialty timbers.

Ecotypes and pairings include, for example, old growth forest, habitat rescue (restoration plants after fires, hurricanes, tornados, natural disasters, floods, etc.), herbs and spice gardens for windowsills and limited space, urban forest, agricultural and garden, optionally with mycopesticides, wildflowers with plants beneficial to bees, birds and/or bat, medicinal plants and mushrooms, native seeds, grasses and spiritual and sacred plants and mushrooms.

Researchers Drew and Smardon et al. at the SUNY College of Environmental Science and Forestry have developed methods and models for choosing the best urban trees to reduce greenhouse gases and increase air quality based on regional climate and the city's typical weather conditions. Carbon sequestration may be increased and the emission of volatile organic compounds may be reduced by planting the right mix of trees. In Syracuse, a Central New York city, the ideal mixture would consist of 31 species of trees, including American basswood, dogwood, Eastern white pine (Pinus strobus), Eastern red cedar, gray birch, red maple and river birch. An ideal selection of trees maximizes carbon sequestration while minimizing the tree's emissions of volatile organic compounds including terpenes and isoprenes, which can increase formation of ozone and exacerbate smog. Factors including species composition, diameter distribution, large size, long life, tree health, species diversity and exotic, non-invasive species vs. native species distribution, disease resistance and native or non-invasive exotic status should be considered. A maximum of 10 percent of any one species, 20 percent of any one genus and 30 percent of any one family is recommended.

For maximum effect, the trees can be planted on barren land or places where natural disasters, including wildfire, have killed off the forest. With forest applications, typically a plurality of tree families, genera and species are preferred, as a single species over a large area reduces biodiversity and environmental stability (particularly a non-native tree with few native plant or animal species adapted to the planted species).

Combinations are preferred for creating a forest having a plurality of trees and fungi, in essence establishing sufficient biodiversity which can lead to a mutually sustainable ecosystem. Preferred tree species include Abies amabilis (Pacific silver fir), Abies balsamea (blue balsam fir), Abies concolor, Abies fraseri (Fraser balsam fir), Abies grandis (grand fir (coastal and interior)), Abies lasiocarpa (alpine fir), Abies magnifica (California red fir), Abies procera (noble fir), Acer rubrum (red maple (northern)), Alnus rubra (red alder), Alnus sinuata (Sitka alder), Acer spicatum (mountain maple), Alnus rhombifolia (white alder), Betula occidentalis (water birch), Betula lenta (sweet birch), Betula lutea (yellow birch), Betula papyrifera (paper birch), Betula populifolia (grey birch), Carpinus caroliniana (American hornbeam), Catalpa speciosa (northern catalpa), Chamaecyparis lawsonia (Port Orford cedar), Chilopsis linearis (desert willow), Cornus nuttallii, Cornus sericea, Crataegus cordata (Washington hawthorn), Crataegus douglassi, Cupressus arizonica (Arizona cypress), Cupressus macrocarpa (Monterey cypress) Fraxinus anomala (desert ash), Juniperus communis, Juniperus scopulorum (Rocky Mountain juniper), Larix laricina (American larch), Larix occidentalis (western larch), Liquidambar styraciflua (sweet gum), Liriodendron tulipifera (tulip poplar (dewinged seeds)), Metasequoia glyptostroboides, Morus rubra (mulberry), Picea breweriana (brewers spruce), Picea engelmanni (Engelman spruce), Picea glauca (white spruce), Picea glauca densata (Black Hills spruce), Picea mariana (black spruce), Picea pungens glauca (blue spruce), Picea rubens (red spruce), Picea sitchensis (Sitka spruce), Pinus albicaulis, Pinus echinata (yellow pine), Pinus contorta contorta (shore pine), Pinus contorta latifolia (lodgepole pine), Pinus glabra (spruce/cedar pine), Pinus monticola (western white pine), Pinus muricata (Bishop pine), Pinus ponderosa (Ponderosa pine), Pinus resinosa (red pine), Pinus serotina (pond pine), Pinus strobus (eastern white pine), Pinus virginiana (Virginia pine), Platanus occidentalis (American sycamore), Populus tremuloides, Prunus emarginata, Prunus virginiana, Pseudotsuga menziesii (Douglas fir (coastal and interior)), Rhus copallina (flameleaf sumac), Rhus glabra, Robina pseudoacacia (black locust), Salix lasiandra, Salix scouleriana, Sambucus glauca (blue elderberry), Sequoia sempervirens (coastal redwood), Sequoiadendron giganteum (giant sequoia), Sorbus americana (American mountain ash), Sorbus scopulina (western mountain ash), Taxus brevifolia, Thuja occidentalis (arborvitae), Thuja plicata (red cedar), Tsuga canadensis (eastern hemlock), Tsuga caroliniana (Carolina hemlock), Tsuga heterophylla (western hemlock), Tsuga mertensiana (mountain hemlock), Ulmus americana (American elm) and Viburnum cassinoides (tea berry). Preferred tree species, for example for the Pacific coast from California to Washington, include, sequoias, redwoods, cedar, alder, birch, yew (Taxus brevifolia (Pacific yew), and other Taxus species), and the family Pinacae, which includes pines, hemlocks, firs and larches. Aspen trees, subalpine firs matched with subalpine grasses, flowers, and fungal spores such as Stropharia riparia, a saprophytic fungus, and Amanita muscaria, a mycorrhizal fungus, would be useful for boxes appropriate for the Rocky Mountain ecosystems. Fruit and apple trees with mycorrhizal fungi in combination with Morel (Morchella species) spores are an example; Morels are also useful in burn areas.

It will be appreciated that all seeds of suitable sizes may be employed with the present invention. With corrugated medium, larger flute sizes will allow for larger sized seeds. Flute size must be balanced against flute characteristics; larger flute profiles provide better vertical compression strength and cushioning, while smaller profiles provide superior resistance to process and printing crush. Typically seeds of two or three millimeters or less in diameter will fit in all but the smallest flute and microflute sizes without any tearing or bulging; one millimeter and smaller will fit all the usual flute sizes (ranging from 1/16 to 5/16 from flute top to flute top). By way of example but not by limitation, the seeds of a garden group of plant species could be selected from the group comprised of onions, carrots, corn, kale, broccoli, mustard, lettuce, cucumbers, wheat, rice, oats, rye, poppies, lentils, beans, squash, melons, potatoes, tomatoes, turnips, garlic, ginger, mustard, chard, cilantro, fennel, oregano, chives, basil, thyme, dill and other garden plants, herbs and spices. Examples of native grass, sedge, rush and grass-like seeds and cultivated seeds include Agrostis exarata (Spike Bentgrass), Ammophila arenaria (European sand dune or beach grass), Ammophila breviligulata (American beach grass), Ammophila champlainensis Seymour, Ammophila maritima, Beckmannia zyzigachne (American Sloughgrass), Bromus carinatus (California Brome), Bromus vulgaris (Columbia Brome), Carex densa (Dense-Headed Sedge), Carex feta (Green-Sheathed Sedge), Carex leporina (Harefoot Sedge), Carex lenticularis (=C. kelloggii) (Shore Sedge), Carex lyngbyel (Lyngby Sedge), Carex macrocephala (Big Headed Sedge), Carex obnupta (Slough Sedge), Carex pansa (Foredune Sedge), Carex unilateralis (One-Sided Sedge), Deschampsia caespitosa (Tufted Hair Grass), Eleocharis palustis (Creeping Spike rush), Elymus glaucus (Blue Wild Rye), Festuca idahoensis var. roemeri (Roemer's Fescue), Festuca rubra var. littoralis (Shore Fescue), Festuca subulata (Bearded Fescue), Glyceria elata (Tall Mannagrass), Glyceriaoccidentalis (Western Mannagrass), Hordeum brachyantherum (Meadow Barley), Juncus effusus (Soft Rush), Juncus patens (Spreading Rush), Juncus tenuis (Slender Rush), Lozula campestris (Woodrush), Phalaris arundinacea (Reed Canary Grass), Phalaris aquatica, Phalaris tuberosa (Staggers Grass), Phalaris canariensis, Poa Macrantha (Dune Bluegrass), ReGreen (Sterile Hybrid Wheat), Scirpus acutus (Hardstem Bullrush), Scirpus americanus, Scirpus cyperinus, Scirpus maritimus (Seacoast Bullrush), Scirpus microcarpus, Scirpus validus, Sparaganuim eurycarpum (Giant Burreed), Triglochin maritinum (Seaside Arrowgrass), Typha latifolia (Cattail), Alopecuris geniculatus, Carex pachystachya, Carex stipata (grass like), Danthonia californica, Eleocharis ovata (grass like), Glycaria grandis, Juncus acuminatus, Juncus bolanderi and Juncus ensifolius (Daggar leaf rush). By way of example, other plants include succulents and cacti, Marijuana (Cannabis indica, Cannabis sativa), Lily of the Nile (Agapanthus africanus), white fountain grass (Pennisetum ruppellii), muhly grass (Muhlenbergia capillaris), African iris (Dietes vegeta), podocarpus (Podocarpus macrophyllus), wax myrtle (Myrica cerifera), Aztec grass (Ophiopogon intermedius argenteomarginatus), mondo grass (Ophiopogon japonicus), evergreen giant (Liriope muscan), evergreen Paspalum (Paspalum quadrifarium) and sand cord grass (Spartina bakerii). Animal specific species or combinations of plants that are desirable to the animals for which the food is destined include catnip, Nepeta cataria, for cats and boxes and corrugated panels customized to include seeds of plants which produce flowers specific to the needs of bees, birds and bats, which spread pollen to the benefit of the ecological community. A catnip seed and spore infused box can be utilized for cat food; a hemp seed and spore box can be used to ship bird food. Moreover, pollen can be added to further enhance a seed and spore infused box to attract pollinating insects.

For use with trees and other slow germinating plants, a cover crop of, for example, grass seeds can be applied in the mixture to give a fast germinating ground cover, the grasses typically germinating first followed by germination of the tree seeds (or native grasses, etc.) being planted established.

Boxes and corrugated panels can be customized to include endophytic, entomopathogenic, mycorrhizal, and saprophytic fungal species which confer host defense of resistance to parasitic organisms, including viruses, bacteria, fungi, algae, insects, and grazing animals. Boxes and corrugated panels can be customized to include endophytic, entomopathogenic, mycorrhizal, and saprophytic fungal species which attract beneficial organisms, including viruses, bacteria, fungi, algae, insects, and grazing animals which help the survival of the habitats biodiversity.

The present invention provides further advantages via use of a fungal component or components in biodegradable materials to help catalyze significant climate change in arid environments through the enhancement of the water retention capacities of the top soils, leading to the ‘oasis’ phenomena in dryland habitats, the net effects of which are not only erosion control, but significant enhancement of biological communities which then can become ‘seed’ banks leading to a creations of satellite communities in proximity to the genome source. More particularly, the judicious placement of such living cardboard panels along the interface peripheries of desert, grassland, and forestland environments can help reverse trends towards desertification.

Another advantage of the present invention is the use of fungal components in biodegradable materials to create communities of fungi, including commercially valuable mushrooms.

Products, processes and business methods utilizing cardboard for seed planting and fungal inoculation makes advantageous use of several fungal characteristics. For example, it has been found by the present inventor that quite different techniques are called for when inoculating soils and non-sterile substrates as compared to sterile substrates. When inoculating sterilized or pasteurized substrates, or materials composted so as to prepare a selective nutritious medium of such characteristics that the growth of mushroom mycelium is promoted to the practical exclusion of competitor organisms (see The Mushroom Cultivator (1983) by Stamets and Chilton), a technique known as “through spawning” is preferable, wherein the fungal inoculum is introduced via numerous inoculation points (such as colonized grain spawn or sawdust spawn) throughout the medium. However, such an approach in non-sterile bulk substrates such as soil or wood chips may lead to die-off of the planted mycelium. Each inoculation point becomes a separate colony surrounded by competitor organisms in all directions, often with the result that the inoculation points are unable to generate the necessary mycelial momentum to successfully colonize the substrate. The present inventor has found “layer spawning” or “sheet inoculation,” wherein the fungal inoculum is spread in a horizontal layer within the non-sterile bulk substrate, to be much more successful. Such sheet inoculation takes advantage of several fungal characteristics: 1) mycelia often grows and spreads most rapidly in the lateral, horizontal directions; 2) when mycelia grows horizontally and links into a mycelial layer or mat, it becomes much more vigorous, resistant to contaminants and competitive, allowing further successful growth and colonization in the vertical direction; and 3) ‘wild’ mycelial organisms are typically matlike and layered in that they may cover many acres, yet be only a few inches deep. Thus a cardboard panel introduces inoculation points and allows for horizontal growth in accord with the mushroom or fungi's natural characteristics.

Virtually all fungi may prove useful in reforestation, agriculture and habitat preservation and restoration. Fungi useful in the present invention include saprophytic fungi (including gilled, polypore and other types of mushrooms), mycorrhizal fungi (which form a mutually dependent, beneficial relationship with the roots of host plants ranging from trees to grasses to agricultural crops, as may certain saprophytic fungi), and fungi imperfecti (those asexually reproducing fungi related to the sexually reproducing “fungi perfecti” or “mushroom fungi”). All fungi and their spores and hyphae should be considered to be a useful part of the invention and may be favorably employed in the appropriate ecosystems.

Either spores or mycelium of saprophytic fungi may be utilized. Mycelium may be metabolically arrested through freeze-drying (flash chilling), air drying, or by other means, for storage, transportation and subsequent rehydration for field deployment. Storage time of up to a year or more is possible.

Suitable mycorrhizal spores include, by way of example but not of limitation, the endomycorrhizal species Glomus aggregatum, Glomus brasilianum, Glomus clarum, Glomus etunicatum, Glomus deserticola, G. intradices, Glomus monosporum, G. mosseae and G. tunincatum and Gigaspora margarita useful with, for example, cedars and redwoods, sequoias, and alders, the ectomycorrhizal species Gomphidius glutinosus, Rhizopogon amylopogon, Rhizopogon fulvigleba, Rhizopogon luteolus, Rhizopogon parksii, Rhizopogon villosullus, Pisolithus tinctorius, Suillus granulatus, Suillus punctatapies, Laccaria bicolor, Laccaria laccata useful with pines, firs and deciduous trees, and Scleroderma, useful with firs, hemlocks and birch. Useful endophytic fungi include Curuularia protuberata, Colleotrichum, Xerula species and, for yews, Taxomyces.

Information on gathering useful and beneficial saprophytic mushrooms for spores or hyphae may be found in standard mycological field guides such as Mushrooms Demystified (1979, 1986) by David Arora, The Audubon Society Field Guide to North American Mushrooms (1981, 1995) by Gary Lincoff and Psilocybin Mushrooms of the World (1996) by Paul Stamets.

Fungal spores may gathered via a variety of means, including but not limited to large scale spore-printing on surfaces and collection from fresh and/or dried mushrooms. A unique method developed by the present inventor is to collect spores from the flexible poly-tubing or other ducting used for distributing air within mushroom growing rooms and mushroom farms. This method is efficient in gathering substantial spore mass.

Mycelial hyphae (including mushrooms, a form of mycelial hyphae) may be cultured using standard mycological techniques for mushrooms. Further information on techniques suitable for production of many of the preferred gourmet, medicinal and ecorestorative mushrooms and their spores and mycelial hyphae may be found in Stamets and Chilton, The Mushroom Cultivator (1983) and Stamets, Growing Gourmet and Medicinal Mushrooms (1993, 2000).

Suitable saprophytic fungal genera include, by way of example but not of limitation, the gilled mushrooms (Agaricales) Agaricus, Agrocybe, Armillaria, Bolbitius, Clitocybe, Collybia, Conocybe, Coprinus, Flammulina, Giganopanus, Gymnopilus, Hypholoma, Inocybe, Hypsizygus, Lentinula, Lentinus, Lenzites, Lepiota, Lepista, Lyophyllum, Macrocybe, Marasmius, Myceliophthora, Mycena, Omphalotus, Panaeolus, Panellus, Pholiota, Pleurotus, Pluteus, Psathyrella, Psilocybe, Schizophyllum, Sparassis, Stropharia, Termitomyces, Tricholoma, Volvaria, Volvariella, etc.; the polypore mushrooms (Polyporaceae) Albatrellus, Antrodia, Bjerkandera, Bondarzewia, Bridgeoporus, Ceriporia, Coltricia, Daedalea, Dentocorticium, Echinodontium, Fistulina, Flavodon, Fomes, Fomitopsis, Ganoderma, Gloeophyllum, Grifola, Hericium, Heterobasidion, Inonotus, Irpex, Laetiporus, Meripilus, Oligoporus, Oxyporus, Phaeolus, Phellinus, Piptoporus, Polyporus, Rigidoporus, Schizopora, Trametes, Wolfiporia, etc.; Basidiomycetes such as Auricularia, Calvatia, Ceriporiopsis, Coniophora, Cyathus, Lycoperdon, Merulius, Phlebia, Serpula, Sparassis and Stereum; Ascomycetes such as Cordyceps, Morchella, Tuber, Peziza, etc.; ‘jelly fungi’ such as Tremella; the mycorrhizal mushrooms (including both gilled and polypore mushrooms) and endomycorrhizal and ectomycorrhizal non-mushroom fungi such as Acaulospora, Alpova, Amanita, Astraeus, Athelia, Boletinellus, Boletus, Cantharellus, Cenococcum, Dentinum, Gigaspora, Glomus, Gomphidius, Hebeloma, Lactarius, Paxillus, Piloderma, Pisolithus, Rhizophagus, Rhizopogon, Rozites, Russula, Sclerocytis, Scleroderma, Scutellospora, Suillus, Tuber, etc.; fungi such as Phanerochaete (including those such as P. chrysosporium with an imperfect state and P. sordida); the fungi imperfecti and related molds and yeasts including Actinomyces, Alternaria, Aspergillus, Botrytis, Candida, Chaetomium, Chrysosporium, Cladosporium, Cryptococccus, Dactylium, Doratomyces (Stysanus), Epicoccum, Fusarium, Geotrichum, Gliocladium, Humicola, Monilia, Mucor, Mycelia Sterilia, Mycogone, Neurospora, Papulospora, Penicillium, Rhizopus, Scopulariopsis, Sepedonium, Streptomyces, Talaromyces, Torula, Trichoderma, Trichothecium, Verticillium, etc.; and entomopathogenic fungi such as Metarhizium, Beauveria, Paecilomyces, Verticillium, Hirsutella, Aspergillus, Akanthomyces, Desmidiospora, Hymenostilbe, Mariannaea, Nomuraea, Paraisaria, Tolypocladium, Spicaria, Botrytis, Rhizopus, the Entomophthoracae and other Phycomycetes, and Cordyceps. It will also be noted that fungi imperfecti, molds and yeasts may produce spores, conidia, perithecia, chlamydospores, etc. and other means of generating progeny. All such fungi imperfecti, molds, yeasts, stages, forms and spores should be considered as suitable for the practice of the present invention.

Suitable fungal species include by way of example only, but not of limitation: Agaricus augustus, A. blazei, A. brasiliensis, A. brunnescens, A. campestris, A. lilaceps, A. placomyces, A. subrufescens and A. sylvicola, Acaulospora delicata; Agrocybe aegerita and A. arvalis; Albatrellus hirtus and A. syringae; Alpova pachyploeus; Amanita muscaria; Antrodia carbonica and A. radiculosa; Armillaria bulbosa, A. gallica, A. matsutake, A. mellea and A. ponderosa; Astraeus hygrometricus; Athelia neuhoffii; Auricularia auricula and A. polytricha; Bjerkandera adusta and B. adusta; Boletinellus merulioides; Boletus punctipes; Bondarzewia berkeleyi; Bridgeoporus nobilissimus; Calvatia gigantea; Cenococcum geophilum; Ceriporia purpurea; Ceriporiopsis subvermispora; Collybia albuminosa and C. tuberosa; Coltricia perennis; Coniophora puteana; Coprinus comatus, C. niveus and ‘Inky Caps’; Cordyceps variabilis, C. facis, C. subsessilis, C. myrmecophila, C. sphecocephala, C. entomorrhiza, C. gracilis, C. militaris, C. washingtonensis, C. melolanthae, C. ravenelii, C. unilateralis, C. clavulata and C. sinensis; Cyathus stercoreus; Daedalea quercina; Dentocorticium sulphurellum; Echinodontium tinctorium; Fistulina hepatica; Flammulina velutipes and F. populicola; Flavodon flavus; Fomes fomentarius; Fomitopsis officinalis and F. pinicola; Ganoderma annularius, G. applanatum, G. australe, G. curtisii, G. japonicum, G. lucidum, G. neo-japonicum, G. oregonense, G. sinense and G. tsugae; Gigaspora gigantia, G. gilmorei, G. heterogama, G. margarita; Gliocladium virens; Gloeophyllum saeparium; Glomus aggregatum, G. calcdonius, G. clarus, G. fasciculatum, G. fasiculatus, G. lamellosum, G. macrocarpum and G. mosseae; Grifola frondosa; Hebeloma anthracophilum and H. crustuliniforme; Hericium abietes, H. coralloides, H. erinaceus and H. capnoides; Heterobasidion annosum; Hypholoma capnoides and H. sublateritium; Hypsizygus ulmarius and H. tessulatus (=H. marmoreus); Inonotus hispidus and I. obliquus; Irpex lacteus; Lactarius deliciosus; Laetiporus sulphureus (=Polyporus sulphureus); Lentinula edodes; Lentinus lepideus, L. giganteus, L. ponderosa, L. squarrosulus and L. tigrinus; Lentinula species; Lenzites betulina; Lepiota rachodes and L. procera; Lepista nuda (=Clitocybe nuda); Lycoperdon lilacinum and L. perlatum; Lyophyllum decastes; Macrocybe crassa; Marasmius oreades; Meripilus giganteus; Merulius incarnatus, M. incrassata and M. tremellosus; Morchella angusticeps, M. crassipes and M. esculenta; Mycena citricolor and M. chlorophos; Omphalotus olearius; Panellus stypticus; Paxillus involutus; Penicillium oxalicium; Phaeolus schweinitzii; Phellinus igniarius P. linteus and P. weirii; Pholiota nameko; Piloderma bicolor, Piptoporus betulinus; Pisolithus tinctorius; Pleurotus citrinopileatus (=P. cornucopiae var. citrinopileatus), P. cystidiosus, (=P. abalonus, P. smithii (?)), P. djamor (=P. flabellatus, P. salmoneo-stramineus), P. dryinus, P. eryngii, P. euosmus, P. ostreatus, P. pulmonarius (=P. sajor-caju) and P. tuberregium; Pluteus cervinus; Polyporus indigenus, P. saporema, P. squamosus, P. tuberaster and P. umbellatus (=Grifola umbellata); Psathyrella hydrophila, Psilocybe aztecorum, P. azurescens, P. baeocystis, P. bohemica, P. caerulescens, P. cubensis, P. cyanescens, P. hoogshagenii, P. mexicana, P. pelliculosa, P. semilanceata, P. tampanensis and P. weilii; Rhizopogon nigrescens, R. roseolus and R. tenuis (=Glomus tenuis); Schizophyllum commune; Schizopora paradoxa; Sclerocytis sisuosa; Serpula lacrymans and S. himantioides; Scleroderma albidum, S. aurantium and S. polyrhizum; Scutellospora calospora; Sparassis crispa and S. herbstii; Stereum complicatum and S. ostrea; Stropharia aeruginosa, S. cyanea, S. albocyanea, S. caerulea and S. rugoso annulata; Suillus cothurnatus; Talaromyces flavus; Termitomyces robustus; Trametes hirsuta, T. suaveolens and T. versicolor, Trichoderma viride, T. harmatum; Tricholoma giganteum and T. magnivelare (Matsutake); Tremella aurantia, T. fuciformis and T. mesenterica; Volvariella volvacea; and numerous other beneficial fungi.

For ecological restoration, all the fungi (including not only economically valuable species but also “little brown mushrooms” and “toadstools”) may play a valuable role, including stump and log dwelling fungi, wood chip dwelling fungi, ground dwelling fungi, mycorrhizal fungi and the fungi imperfecti. For example, spores or hyphae of the genus Morchella such as Morchella angusticeps, M. crassipes and M. esculenta, gourmet ground dwelling mushrooms and trees such as poplar that are known to favor fire-burned areas, may optionally be utilized in the present inventions in fire recovery efforts, thereby introducing a potential source of very rapidly growing mycelium into the soil at the same time seeds are introduced. Preferred species for ecological restoration (and most other purposes) include Auricularia polytricha; Agaricus blazei and A. brunnescens; Agrocybe aegerita; Bridgeoporus nobilissimus; Coprinus comatus; Flammulina velutipes and F. populicola; Fomes fomentarius; Fomitopsis officinalis and F. pinicola; Ganoderma lucidum, G. oregonense and G. tsugae; Grifola frondosa; Hericium abietes and H. erinaceus, Hypholoma capnoides and H. sublateritium; Hypsizygus ulmarius and H. tessulatus; Laetiporus sulphureus; Lentinula edodes; Lepista nuda; Morchella angusticeps; Pholiota nameko; Pleurotus citrinopileatus, P. cystidiosus, P. eryngii, P. euosmus, P. ostreatus, P. pulmonarius and P. tuberregium; Polyporus umbellatus and P. tuberaster; Psilocybe azurescens, P. cubensis, P. cyanescens, P. mexicana, P. semilanceata and P. tampanensis (where these species are legal for such purposes); Sparassis crispa; Stropharia rugoso annulata; Trametes versicolor; Tremella fuciformis; and Volvariella volvacea.

A single species may be employed for a single application—for example, a single saprophytic species on a fiber substrate in conjunction with a single plant species such as Hypsizygus ulmarius on sawdust with corn. For typical ecological restoration, mycoremediation of toxic wastes, and particularly habitat restoration and reforestation, etc., a plurality of species is preferred. The variety of species produce different species specific enzymatic systems that break down different chemicals and make these chemicals biologically available as nutrients for the microsphere and the biosphere. An example can be seen in the breakdown of a recalcitrant substrate—a hardwood such as ironwood, a substrate containing high concentrations of the complex polyaromatic cellulose carbohydrate compounds and the complex heterogeneous polyaromatic polymer lignin. A succession of mushrooms may be grown on the same wood, each species breaking down different compounds via different enzymatic systems, thereby making the carbon, nitrogen, phosphorus, hydrogen, etc. available as nutrients. To illustrate, a succession of gourmet mushroom species may be grown on the same wood. For example, Lentinula edodes (Shiitake) may be first grown on the wood, then Pleurotus ostreatus (Oyster), then Stropharia rugoso annulata (King Stropharia, Garden Giant or ‘Godzilla Mushrooms’), at which point the wood will have been transformed into a rich soil, suitable for gourmet mushrooms such as Coprinus comatus (Shaggy Mane). The same principle can be observed in nature where three or four different mushroom species may be observed fruiting from the same stump, each digesting a different woody compound and making the compounds available to the biosphere in the form of mycelium and mushrooms, or where different species of mushrooms may be observed fruiting from the floor of the forest adjacent to each other. The saprophytic mushrooms illustrated above also make such nutrients available to mycorrhizal fungi, thus further enhancing the symbiotic relationship with plants and resulting in greatly increased growth. Thus a plurality of fungal strains and species is most preferred. Preferred species for mycoremediation include the saprophytic mushrooms Fomes fomentarius, Pleurotus ostreatus and Trametes versicolor (E. Coli and other bacteria, protists, pathogens etc.); Fomitopsis pinicola; Ganoderma lucidum, G. oregonense and G. tsugae; Laetiporus sulphureus; Pleurotus ostreatus and the other Pleurotus species (oils, polyaromatic, alkane and alkene hydrocarbons including chlorinated compounds, brominated compounds, hormones, etc.); Polyporus umbellatus (malaria and other bacteria); Psilocybe azurescens and P. cyanescens (Sarin and VX and other phosphorylated nerve gases, organophosphate pesticides, etc.); Stropharia rugoso annulata (bacteria, urban and agricultural runoff, mycofiltration of silts, bacteria, bacteriophages, viruses), as a “follow-up” species to Pleurotus and other white-rot fungi, etc.); and Trametes versicolor and other Trametes and species (Sarin, VX and other phosphorylated nerve gases, organophosphate pesticides, etc.), Collybia and the similar Marasmius and numerous “satellite genera” (metals, heavy metals, ores, etc.) as well as the other gilled and polypore genera and species listed above.

The present invention provides further advantages through use of entomopathogenic fungal components to control, reduce or eliminate pest insects or disease-carrying insects in the applied environments. More broadly, fungal components in biodegradable materials may be utilized to control harmful insects, enhance insect communities, or invite beneficial insects in the applied environments. Since insect communities can influence or predetermine bird and bat communities, the fungal constituent has a direct downstream effect on this and many other biological successions.

Of particular use where insect pest control is desired are the entomopathogenic fungi Metarhizium, Beauveria, Cordyceps, Paecilomyces, Verticillium, Hirsutella and Aspergillus including Metarhizium anisopliae, Metarhizium flaviride, Beauveria bassiana, Beauveria brongniartii, Beauveria amorpha, Pacilomyces fumosoroseus, Verticillium lecanii, Hirsutella citriformis, Hirsutella thompsoni, Cordyceps variabilis, Cordyceps facis, Cordyceps subsessilis, Cordyceps myrmecophila, Cordyceps sphecocephala, Cordyceps entomorrhiza, Cordyceps gracilis, Cordyceps militaris, Cordyceps washingtonensis, Cordyceps melolanthae, Cordyceps ravenelii, Cordyceps unilateralis, Cordyceps clavulata and Aspergillus flavus. In addition to known uses of spores, the preconidial mycelium of entomopathogenic fungi has been found to be attractant and/or pesticidal to such pest insects as termites, fire ants, carpenter ants, fungus gnats, etc. See U.S. Pat. No. 6,660,290 (2003) for Mycopesticides and U.S. Pat. No. 7,122,176 (2006) for Mycoattractants and Mycopesticides, both to Stamets, and both incorporated in their entirety by reference.

Insect pest control benefits are also provided by mycorrhizal fungi. Plants infected by endophytic fungi are known to be chemically protected against consumption by insect pests, for example aphids. Insect herbivore-parasite interaction webs on endophyte-free grasses show enhanced insect abundance at alternate trophic levels, higher rates of parasitism and increased dominance by a few trophic links, whereas plants infected with endophytes alter insect herbivore abundance, selectively favoring beneficial insects and higher organisms. It is conceivable that the effect of plant endosymbionts on food webs will cascade up through various trophic pathways and can mediate competitive interactions between plant species affecting vegetation diversity and succession. Ornacini et. al., Symbiotic fungal endophytes control insect host-parasite interaction webs, Nature, 409: 78-81 (4 Jan. 2001). Thus in addition to their direct symbiotic effects benefiting plants, it is expected that mycorrhizal fungi can reduce pest insect herbivores, thus favoring beneficial insects and higher organisms and thereby increasing biodiversity.

The present invention utilizes the design and active insertion of individual saprophytic, mycorrhizal, entomopathogenic, and parasitic fungal species and mosaics of species to catalyze habitat recoveries from catastrophia. Furthermore, by using delivery systems and mycotechnologies disclosed herein instead of relying on serendipitous sporefalls, environmental designers can greatly benefit by establishing, strengthening or steering the course of habitat evolution in a fashion that is both environmentally sound and/or economically profitable. In installing new parks, landscapes, forests, arboretums, green roofs, habitat oases and oasis-islands, the insertion of purposely designed ‘fungal footprints’ can dramatically improve the biodynamics of any ecosystem.

Inoculation of cardboard with beneficial fungal spores and/or mycelial hyphae and seeds provides products and methods useful for purposes including enhancing plant growth and mycorrhizal and symbiotic relationships, habitat restoration, erosion control and stabilization of soils, treatment of contaminated habitats, filtration (“mycofiltration”) of agricultural and urban water runoff, fungal bioremediation (“mycoremediation”) of biological and chemical pollutants and toxic wastes, and production of mycelia and mushrooms and improved production of plants, providing nutrients to insects, herbivores and numerous organisms up and down the food chain as well as generating carbon credits.

The present business methods, processes and compositions utilizing cardboard/seeds/spores can also be economically applied, for example, when used for: 1) Habitat recovery/reclamation including ‘regreening’ of roads, especially logging roads, back into native ecosystems or wilderness; 2) Mycofiltration and prevention of sediment and silt runoff into waterways from existing logging or gravel roads, depleted environments, scarred, burned or biologically hostile environments. The mycelium retains sediments and silts, incorporating them into topsoil for tree growth while preventing release into waterways. Such fungally colonized and seeded cardboard products protect sensitive watersheds such as salmon spawning grounds, providing mushroom and mycelial biomass which then feed developing larvae of numerous insects which benefit fisheries through enhancement of the food chain and from protection from upland runoff; 3) Protection of sensitive watersheds and ecosystems from upland or neighboring sources/vectors of biological or chemical contamination by capture and mycoremediation in the mycelial network. This is critical for urban developments, protection of salmon or trout streams, estuary environments, etc. Sediment and silt runoff into salmon and trout spawning grounds are known to create environment hostile to egg survival. Similar negative habitat effects result from runoff into other bodies of water. By utilizing mycofiltration, the silt and sediment becomes part of a rich soil as opposed to a marine pollutant; 4) Environmental and agricultural enhancement and control of pest microorganisms and insects. Harmful biological organisms that can be digested and destroyed by fungal mycelia include viruses, bacteria, protozoa, nematodes, rotifers and insect pests. 5) Reduction for the need for fertilizers, water and outside inputs that are needed to create, maintain, and sustain green roofs on buildings and other urban and suburban greenbelt zones and green architectures. Thus by infusing fungal inoculant into cardboard, targeted organisms such as bacteria, fungi, viruses, protozoa, rotifers, amoebas, disease carrying or ‘nuisance’ insects and their larvae, and nematodes can be effectively reduced where such is a problem. Control of plant pathogens such as Rhizoctonia solani, Sclerotium rolfsii, Verticillium dahliae and other soilborne plant diseases may also be provided by saprophytic and mycorrhizal fungi and by fungi imperfecti such as Trichoderma viride, T. harmatum and Gliocladium virens. Endophytic fungi (i.e., Curvularia and Colleotrichum), mycorrhizal and saprophytic species have anti-fungal properties against Aspergillus and other aggressive pathogenic-to-plant fungi; 6) Planting of poplars, cottonwoods and other trees for hydraulic control and protection of groundwater; 7) Controlling social insects such as fire ants, carpenter ants and termites utilizing pre-conidial mycelia of mycopesticidal, entomopathogenic fungi on cardboard. As the mycelia grows, it also outgases attractant fragrances. The insect consumes and otherwise makes contact with fragments of mycelia. As the insect travels, mycelia is spread. As the insect weakens with illness, the mycelia manifests, becoming more pervasive and stronger. The insect is killed by infectious colonization of the fungus. The time delay of exposure to death is an added advantage as it allows the infected individuals to fully disperse through the affected region as well as the nest without being sequestered and expunged from the colony. See U.S. Pat. Nos. 6,660,290 (2003) and 7,122,176 (2006) to Stamets, herein incorporated in their entirety by reference; 8) The growth of algae in ponds and lakes can be directly attributed to the phosphorus-rich runoff from agricultural fertilizers and other industrial pollutants. Phosphorus is typically the ‘limiting nutrient’ of algae growth. By removing phosphorus using cardboard inoculated with dephosphorylating fungi such as Trametes versicolor, Psilocybe azurescens, and others, the over-growth algae can be limited in lakes and ponds, providing cost and ecological saving benefits to fishery ecologies and the watershed. A similar approach may be employed in those soils contaminated with organophosphate pesticide residues. The cardboard may be infused with the mycelia of anti-microbial fungi such as Fomes fomentarius, Fomitopsis officinalis, Ganoderma applanatum, Ganoderma oregonense, Trametes versicolor, Lentinula edodes, Laetiporus sulphureus, Pleurotus eryngii, Pleurotus ostreatus, Polyporus umbellatus, Psilocybe semilanceata, Schizophyllum commune, Stropharia rugoso annulata, and Calvatia species; 9) Mycofiltration of pesticides, including both organophosphate and halogenated pesticides, which are thought in minute quantities to interfere with salmon's olfactory sense, thereby impeding the return to breeding grounds and successful reproduction. The present invention as described herein may be effectively employed to reduce, ameliorate, limit or prevent the impact of pesticides and other agricultural and/or urban contaminants upon riparian habitats and marine environments and the associated fisheries, recreational use, drinking water, etc.: 10) Wide scale inoculation of gourmet and medicinal mushroom species for use in various agricultural, forestry, ecological and bioremediation purposes. Gourmet and medicinal mushrooms containing valuable physiologically active compounds and pro-compounds and valuable enzymes, enzyme precursors and useful chemical compounds may be utilized in the cardboard. Fungal species may be selected for a specific environment, for example lawns, gardens, crop fields, forests (ranging from plains to mountainous to tropical ecosystems environments) and aquatic environments including riparian, marsh, wetlands, estuaries, ponds, lakes, ditches and saline environments. By selecting the type of fungi, an ecologist, remediator, forester, farmer, landscaper, ecological designer, astrobiologist, architect and others can direct the course of ecological recovery or ecological preservation, thereby improving the economical usefulness of the land for varying forest, farm, riparian, agricultural and urban uses; 11) Stabilization of soils. For example, the tenacity of Ammophila maritima, a dune grass planted by the Army Corp of Engineers to prevent jetty erosion around the Columbia River as it enters the Pacific Ocean, is significantly enhanced through the domination of the mycelium of Psilocybe azurescens and P. cyanescens in the top soils of that biosphere. Soil structure, pH and fertility is improved; 12) Boxes for assisting refugees, indigenous displaced peoples, including victims from natural and man-made disasters. As the first emergency relief often is delivered to refugees in a box, there is the economically feasible opportunity of utilizing the delivery box as inoculum for growing plants and fungi. The insides of the box could be sorted according to species of plants, climatic zones, pH requirements, and soil conditions. Such box panels would be recognized by the recipients as having a value, a natural currency for anyone who has an interest in cultivating and habitat recovery. The educational and ecological lesson from having children using the ‘living box’ is as important an advantage of this invention as any aspect previously described; 13) Colleotrichum species are endophytic fungi which may potentiate the antimicrobial properties resident within many Artemisia (worm wood). Artemesia annua has anti-malarial properties. Boxes infused with worm wood scrub seeds (Artemisia annua and other species of Artemisia, approximately 12,000 seeds per gram) and endophytic fungi such as Colleotrichum species can be useful for delivering goods to areas of the world inflicted with malaria (Plasmodium species including P. falciparum) and other disease causing organisms that are sensitive to the antimicrobial properties of the combination of worm wood seeds and their associated endophytic fungi; 14) Fungal spores and seeds of species known to decompose hydrocarbon based pollutants can be infused into cardboard containers used to ship products such as oils, toxic chemicals, and other potential pollutants, whereby the container carrying these products can be germinated and decompose these pollutants subsequent to leaking of the carried pollutants; 15) Cardboard containers, infused with fungal spores and seeds, may be utilized or shredded containers may be added to hydromulch and sprayed; and 16) The establishment of habitats in space colonies and the colonization of other planets. Seeds, spores and cardboard can be economically transported via drone or spaceship to the targeted planetary body or space station. Their low weight/mass makes them economically attractive bio-cargo for transportation through interplanetary and interstellar space. The importance of fungi as a keystone species with the ability to digest or mineralize rocks makes them essential in soil creation and any self-sustaining habitat. For further examples of mycotechnologies, see Stamets, P. 2005. Mycelium Running: How Mushrooms Can Help Save the World. Berkeley, Calif.: Ten Speed Press, for further advantages.

A major advantage of the present invention is the active adsorption of atmospheric carbon dioxide through sequestering of carbon into the mycelial network and plant life within the soil matrix. Thus, fungal growth, plants and trees can ‘bank-roll’ the carbon credit system through repairing threatened ecosystems by designing the insertion of keystone fungi and plants most beneficial to targeted environmental goals. Fungi retain approximately 50% of the carbon they absorb into their cell walls from enzymatic breakdown of plants and animals. Thicker carbon-rich humus layers support more diverse food chains and life cycles, especially in the descendant plants that subsequently absorb carbon dioxide and respire oxygen. By incorporating carbon into humus and other materials, the carrying capacity of habitats is fortified, increasing the value of the carbon credit. The cardboard, plants and fungi of the present invention provide not only a cost effective method of carbon sequestration, but also the numerous advantages arising from the return of complex biological systems.

### EXAMPLE 1

Double face corrugated cardboard with one face being paper was observed to give superior results as compared to single face sheets with germinating seeds on open corrugated flutes, both the paper and the open corrugations facing up. When moist soil was placed on top, those with a cover paper overgrew with saprophytic fungus and the sprout pushed up through, and more vigorous sprouts emerged as compared with the open corrugations.

### EXAMPLE 2

Mix seeds of Alnus sinuata (Sitka alder), Betula papyrifera (paper birch), Picea sitchensis (Sitka spruce), Pseudotsuga menziesii (Douglas Fir), Sequoia sempervirens (coastal redwood), Sequoiadendron giganteum (giant sequoia) and Thuja plicata (red cedar) and treat to 7-120 days of cold moist stratification. Mix the cold-treated seeds with the seeds of Prunus emarginata, Prunus virginiana, Pseudotsuga menziesii, Rhus glabra, Salix lasiandra and Salix scouleriana. Treat the seed mixture with the spores of the endomycorrhizae Glomus intraradices, Glomus mosseae, Glomus aggregatum, Glomus etunicatum, Glomus deserticola, Glomus monosporum, Glomus clarum, Glomus brasilianum and Gigaspora margarita, the spores of the Ectomycorrhizae Rhizopogon villosullus, Rhizopogon luteolus, Rhizopogon amylopogon, Rhizopogon fulvigleba, Pisolithus tinctorius, Suillus granulatus, Suillus punctatapies, Laccaria bicolor and Laccaria laccata, the saprophytic fungi Reishi (Ganoderma lucidum), Maitake (Grifola frondosa), Pearl Oyster (Pleurotus ostreatus), Conifer Coral (Hericium abietis), Phoenix Oyster (Pleurotus pulmonarius), Turkey Tail (Trametes versicolor) and Shiitake (Lentinula edodes), Trichoderma spp., yeast (Saccharomyces cervisiae) and the bacteria Bacillus subtillus, B. licheniformis, B. azotoformans, B. megaterium, B. coagulans, B. pumlis, B. thurengiensis, B. stearothermiphilis, Paenibacillus polymyxa, P. gordonae, P. durum, Axobacter polymyxa, A. chroococcum, Streptomyces griseues, S. lydicus, Pseudomonas aureofaceans, P florescence and Deinococcus erythromyxa. Incorporate the seeds and spores into cardboard boxes, either corrugated or non-corrugated. Deliver boxes carrying goods to customers with instructions to germinate and carbon credit application form and registration for verification and certification.

### EXAMPLE 3

Cold stratify or heat treat, as appropriate, Abies grandis (grand fir (coast)), Abies grandis (grand fir (interior)), Abies lasiocarpa (alpine fir), Alnus rubra (red alder), Alnus sinuata (Sitka alder), Betula papyrifera (paper birch), Cupressus macrocarpa (Monterey cypress), Picea sitchensis (Sitka spruce), Pinus contorta contorta (shore pine), Pinus contorta latifolia (lodgepole pine), Pinus monticola (western white pine), Pseudotsuga menziesii (Douglas fir (coastal)), Pseudotsuga menziesii (Douglas fir (interior)), Thuja plicata (red cedar), Tsuga heterophylla (western hemlock) and Tsuga mertensiana (mountain hemlock) seeds. Treat the seed mixture with the spores of the endomycorrhizae Glomus intraradices, Glomus mosseae, Glomus aggregatum, Glomus etunicatum, Glomus deserticola, Glomus monosporum, Glomus clarum, Glomus brasilianum and Gigaspora margarita, the spores of the Ectomycorrhizae Rhizopogon villosullus, Rhizopogon luteolus, Rhizopogon amylopogon, Rhizopogon fulvigleba, Pisolithus tinctorius, Suillus granulatus, Suillus punctatapies, Laccaria bicolor and Laccaria laccata, the saprophytic fungi Reishi (Ganoderma lucidum), Maitake (Grifola frondosa), Pearl Oyster (Pleurotus ostreatus), Conifer Coral (Hericium abietis), Phoenix Oyster (Pleurotus pulmonarius), Turkey Tail (Trametes versicolor) and Shiitake (Lentinula edodes), Trichoderma spp., yeast (Saccharomyces cervisiae) and the bacteria Bacillus subtillus, B. licheniformis, B. azotoformans, B. megaterium, B. coagulans, B. pumlis, B. thurengiensis, B. stearothermiphilis, Paenibacillus polymyxa, P. gordonae, P. durum, Axobacter polymyxa, A. chroococcum, Streptomyces griseues, S. lydicus, Pseudomonas aureofaceans, P florescence and Deinococcus erythromyxa. Infuse and incorporate the seeds and spores into cardboard boxes, either corrugated or non-corrugated. Deliver boxes carrying goods to customers with instructions to germinate and carbon credit application form and registration for verification and certification.

### EXAMPLE 4

To activate the recipient removes the goods, and the now empty box is ready for creating a seedling nursery. Since the bottom flat panel is infused with seeds paired with beneficial spores, soil is placed directly into the box to a depth typically of ¼ to 2 inches in depth, and heavily saturated. Water is added as necessary over the course of several weeks until germination is evident as seen from the emergence of young plants. Once the young seedlings have emerged, and depending upon species, the young plant starts are separated and placed into appropriate pots, trays, other containers or directly into the ground, paying particular attention to the needs of the plant species and matching other conditions necessary for their successful maturation.

### EXAMPLE 5

Construct laminated board of recycled products with spores and seeds laminated in the middle of two sheets of 30 pt. uncated recycled board (“URB” or “chip”) or whiteline recycled or two sheets of 20 pt. URB or clay coated recycled board (“CRB”).

It should be understood the foregoing detailed description is for purposes of illustration rather than limitation of the scope of protection accorded this invention, and therefore the description should be considered illustrative, not exhaustive. The scope of protection is to be measured as broadly as the invention permits. While the invention has been described in connection with preferred embodiments, it will be understood that there is no intention to limit the invention to those embodiments. On the contrary, it will be appreciated that those skilled in the art, upon attaining an understanding of the invention, may readily conceive of alterations to, modifications of, and equivalents to the preferred embodiments without departing from the principles of the invention, and it is intended to cover all these alternatives, modifications and equivalents. Accordingly, the scope of the present invention should be assessed as that of the appended claims and any equivalents falling within the true spirit and scope of the invention.

I claim:

1. A business method for packaging and shipping goods and generating carbon credits comprising packaging the goods in a corrugated cardboard container infused with seeds and a fungal inoculant of saprophytic fungi and mycorrhizal fungi, shipping the goods in the corrugated cardboard container via a delivery service and, after delivery of goods, placing the corrugated cardboard container in dirt and watering, whereby, upon growth of the seeds and fungi, carbon is sequestered and carbon credits are generated.

2. The business method of claim 1 wherein the corrugated cardboard container is a cardboard box.

3. The business method of claim 1 wherein the corrugated cardboard container is a container selected from the group consisting of cartons, holders, boxes, overslips, overwrappers, envelopes and retail display cases.

4. The business method of claim 1 wherein the fungal inoculant is selected from the group consisting of spores, mycelium, powdered mushrooms and combinations thereof.

5. The business method of claim 1 wherein a legal entity selected from the group consisting of timber company, paperboard manufacturer, corrugated cardboard manufacturer, box manufacturer, wholesaler, retailer, distributor, box buyer, shipper, delivery company, consumer customer and combinations thereof receive a portion of the carbon credits.

6. The business method of claim 1 wherein a carbon credit application accompanies the corrugated cardboard container.

7. The business method of claim 1 wherein the carbon credits are a form of ecological currency that may be redeemed for benefits selected from the group consisting of cash, carbon tax credits, income tax credits, gas tax credits, sales tax credits, reduction of pollution related tariffs and fines, tax deductions, manufacturer credits, manufacturer rebates and combinations thereof.

8. The business method of claim 1 wherein the seeds are selected from the group consisting of Abies amabilis (Pacific silver fir), Abies balsamea (blue balsam fir), Abies concolor, Abies fraseri (Fraser balsam fir), Abies grandis (grand fir (coastal and interior)), Abies lasiocarpa (alpine fir), Abies magnifica (California red fir), Abies procera (noble fir), Acer rubrum (red maple (northern)), Alnus rubra (red alder), Alnus sinuata (Sitka alder), Acer spicatum (mountain maple), Alnus rhombifolia (white alder), Betula occidentalis (water birch), Betula lenta (sweet birch), Betula lutea (yellow birch), Betula papyrifera (paper birch), Betula populifolia (grey birch), Carpinus caroliniana (American hornbeam), Catalpa speciosa (northern catalpa), Chamaecyparis lawsonia (Port Orford cedar), Chilopsis linearis (desert willow), Cornus nuttallii, Cornus sericea, Crataegus cordata (Washington hawthorn), Crataegus douglassi, Cupressus arizonica (Arizona cypress), Cupressus macrocarpa (Monterey cypress) Fraxinus anomala (desert ash), Juniperus communis, Juniperus scopulorum (Rocky Mountain juniper), Larix laricina (American larch), Larix occidentalis (western larch), Liquidambar styraciflua (sweet gum), Liriodendron tulipifera (tulip poplar (dewinged seeds)), Metasequoia glyptostroboides, Morus rubra (mulberry), Picea breweriana (brewers spruce), Picea engelmanni (Engelman spruce), Picea glauca (white spruce), Picea glauca densata (Black Hills spruce), Picea mariana (black spruce), Picea pungens glauca (blue spruce), Picea rubens (red spruce), Picea sitchensis (Sitka spruce), Pinus albicaulis, Pinus echinata (yellow pine), Pinus contorta contorta (shore pine), Pinus contorta latifolia (lodgepole pine), Pinus glabra (spruce/cedar pine), Pinus monticola (western white pine), Pinus muricata (Bishop pine), Pinus ponderosa (Ponderosa pine), Pinus resinosa (red pine), Pinus serotina (pond pine), Pinus strobus (eastern white pine), Pinus virginiana (Virginia pine), Platanus occidentalis (American sycamore), Populus tremuloides, Prunus emarginata, Prunus virginiana, Pseudotsuga menziesii (Douglas fir (coastal and interior)), Rhus copallina (flameleaf sumac), Rhus glabra, Robina pseudoacacia (black locust), Salix lasiandra, Salix scouleriana, Sambucus glauca (blue elderberry), Sequoia sempervirens (coastal redwood), Sequoiadendron giganteum (giant sequoia), Sorbus americana (American mountain ash), Sorbus scopulina (western mountain ash), Taxus brevifolia, Thuja occidentalis (arborvitae), Thuja plicata (red cedar), Tsuga canadensis (eastern hemlock), Tsuga caroliniana (Carolina hemlock), Tsuga heterophylla (western hemlock), Tsuga mertensiana (mountain hemlock), Ulmus americana (American elm) Viburnum cassinoides (tea berry), onions, carrots, corn, kale, broccoli, mustard, lettuce, cucumbers, wheat, rice, oats, rye, poppies, lentils, beans, squash, melons, potatoes, tomatoes, turnips, garlic, ginger, mustard, chard, cilantro, fennel, oregano, chives, basil, thyme, dill, Agrostis exarata (Spike Bentgrass), Ammophila arenaria (European sand dune or beach grass), Ammophila breviligulata (American beach grass), Ammophila champlainensis Seymour, Ammophila maritima, Beckmannia zyzigachne (American Sloughgrass), Bromus carinatus (California Brome), Bromus vulgaris (Columbia Brome), Carex densa (Dense-Headed Sedge), Carex feta (Green-Sheathed Sedge), Carex leporina (Harefoot Sedge), Carex lenticularis (=C. kelloggii) (Shore Sedge), Carex lyngbyel (Lyngby Sedge), Carex macrocephala (Big Headed Sedge), Carex obnupta (Slough Sedge), Carex pansa (Foredune Sedge), Carex unilateralis (One-Sided Sedge), Deschampsia caespitosa (Tufted Hair Grass), Eleocharis palustis (Creeping Spike rush), Elymus glaucus (Blue Wild Rye), Festuca idahoensis var. roemeri (Roemer's Fescue), Festuca rubra var. littoralis (Shore Fescue), Festuca subulata (Bearded Fescue), Glyceria elata (Tall Mannagrass), Glyceriaoccidentalis (Western Mannagrass), Hordeum brachyantherum (Meadow Barley), Juncus effusus (Soft Rush), Juncus patens (Spreading Rush), Juncus tenuis (Slender Rush), Lozula campestris (Woodrush), Phalaris arundinacea (Reed Canary Grass), Phalaris aquatica, Phalaris tuberosa (Staggers Grass), Phalaris canariensis, Poa Macrantha (Dune Bluegrass), ReGreen (Sterile Hybrid Wheat), Scirpus acutus (Hardstem Bullrush), Scirpus americanus, Scirpus cyperinus, Scirpus maritimus (Seacoast Bullrush), Scirpus microcarpus, Scirpus validus, Sparaganuim eurycarpum (Giant Burreed), Triglochin maritinum (Seaside Arrowgrass), Typha latifolia (Cattail), Alopecuris geniculatus, Carex pachystachya, Carex stipata (grass like), Danthonia californica, Eleocharis ovata (grass like), Glycaria grandis, Juncus acuminatus, Juncus bolanderi and Juncus ensifolius (Daggar leaf rush), succulents and cacti, Marijuana (Cannabis indica, Cannabis sativa), Lily of the Nile (Agapanthus africanus), white fountain grass (Pennisetum ruppellii), muhly grass (Muhlenbergia capillaris), African iris (Dietes vegeta), podocarpus (Podocarpus macrophyllus), wax myrtle (Myrica cerifera), Aztec grass (Ophiopogon intermedius argenteomarginatus), mondo grass (Ophiopogon japonicus), evergreen giant (Liriope muscari), evergreen Paspalum (Paspalum quadrifarium) and sand cord grass (Spartina bakerii) and combinations thereof; the mycorrhizal fungi are selected from the group consisting of Glomus aggregatum, G. brasilianum, G. clarum, G. etunicatum, G. deserticola, G. intradices, G. monosporum, G. mosseae and G. tunincatum, Gigaspora margarita, Rhizopogon amylopogon, R. fulvigleba, R. luteolus, R. parksii, R. villosullus, Pisolithus tinctorius, Suillus granulatus, S. punctatapies, Laccaria bicolor, L. laccata, Scleroderma, Acaulospora, Alpova, Amanita, Astraeus, Athelia, Boletinellus, Boletus, Cantharellus, Cenococcum, Dentinum, Gigaspora, Glomus, Gomphidius, Hebeloma, Lactarius, Paxillus, Piloderma, Pisolithus, Rhizophagus, Rhizopogon, Rozites, Russula, Sclerocytis, Scleroderma, Scutellospora, Suillus, Tuber and combinations thereof; the saprophytic fungi are selected from the group consisting of gilled mushrooms (Agaricales) Agaricus, Agrocybe, Armillaria, Bolbitius, Clitocybe, Collybia, Conocybe, Coprinus, Flammulina, Giganopanus, Gymnopilus, Hypholoma, Inocybe, Hypsizygus, Lentinula, Lentinus, Lenzites, Lepiota, Lepista, Lyophyllum, Macrocybe, Marasmius, Myceliophthora, Mycena, Omphalotus, Panaeolus, Panellus, Pholiota, Pleurotus, Pluteus, Psathyrella, Psilocybe, Schizophyllum, Sparassis, Stropharia, Termitomyces, Tricholoma, Volvaria and Volvariella, polypore mushrooms (Polyporaceae) Albatrellus, Antrodia, Bjerkandera, Bondarzewia, Bridgeoporus, Ceriporia, Coltricia, Daedalea, Dentocorticium, Echinodontium, Fistulina, Flavodon, Fomes, Fomitopsis, Ganoderma, Gloeophyllum, Grifola, Hericium, Heterobasidion, Inonotus, Irpex, Laetiporus, Meripilus, Oligoporus, Oxyporus, Phaeolus, Phellinus, Piptoporus, Polyporus, Rigidoporus, Schizopora, Trametes and Wolfiporia, Basidiomycetes Auricularia, Calvatia, Ceriporiopsis, Coniophora, Cyathus, Lycoperdon, Merulius, Phlebia, Serpula, Sparassis and Stereum, Ascomycetes Cordyceps, Morchella, Tuber and Peziza, jelly fungi Tremella, and saprophytic fungi with an imperfect state such as Phanerochaete chrysosporium and P. sordida.

9. The business method of claim 1 wherein the seeds and fungal inoculant infused into the corrugated cardboard container are selected based on ecological profiles as determined by postal zip codes of shipping destinations.

10. The business method of claim 1 wherein the seeds and fungal inoculant are specifically chosen to help recovery of endangered ecosystems at the destinations to which the corrugated cardboard container are shipped.

11. A business method for shipping goods, generating forest growth and sequestering carbon comprising manufacturing a corrugated cardboard shipping container with seeds and saprophytic and mycorrhizal fungal spores imbedded in the corrugated cardboard, selling the shipping container to a party who utilizes the shipping container to package and ship goods to a consumer who plants the container of cardboard to generate forest growth, sequester carbon and thereby offset global warming and, optionally, generate carbon credits.

12. The business method of claim 11 wherein the seeds are seeds of plants selected from the group consisting of vegetables, cereal crops, agricultural crops, fruits, herbs, spices, trees, shrubs, bushes and combinations thereof.

13. The business method of claim 12 wherein an interactive website records and tracks location, survival rates, growth and carbon mass of trees grown from the seeds.

14. The business method of claim 12 wherein the location, survival rates, growth and carbon mass of trees is verified by satellite imaging.

15. The business method of claim 12 wherein an interactive website allows verification of location, survival rates and growth of trees via satellite imaging.

16. The business method of claim 11 wherein the corrugated cardboard shipping container is a cardboard box.

17. The business method of claim 11 wherein the corrugated cardboard shipping container is a container selected from the group consisting of cartons, holders, boxes, overslips, overwrappers, envelopes and retail display cases.

18. The business method of claim 11 wherein seeds of plants and spores of fungi known to decompose hydrocarbon based pollutants are imbedded in corrugated cardboard shipping containers used to ship oils, toxic chemicals and potential pollutants, whereby the container carrying these products can be germinated and decompose these pollutants subsequent to leaking of shipped hydrocarbon pollutants.

19. A process utilizing corrugated cardboard boxes to produce carbon-absorbing plants and fungi to sequester carbon and combat global warming comprising incorporating seeds, including tree seeds, and saprophytic and mycorrhizal fungal spores into corrugated cardboard, utilizing the corrugated cardboard to manufacture a corrugated cardboard box, said cardboard box being utilized to package goods, the goods then being shipped to a consumer, who then plants the corrugated box and germinates the seeds and fungal spores in the cardboard box, resulting in growth of carbon-absorbing plants and fungi.

20. The process of claim 19 wherein the consumer can thereby offset global warming and qualify for carbon credits.

21. The process of claim 19 wherein the corrugated cardboard boxes qualify for value on carbon credit exchanges as trees mature and absorb carbon.

22. The process of claim 19 wherein the consumer plants the corrugated cardboard box by shipping to a person who germinates the cardboard box and cares for the resulting trees.

23. The process of claim 19 wherein the container additionally comprises spores of endophytic fungi selected from the group consisting of Curvularia protuberata, Colleotrichum, Xerula, Taxomyces and combinations thereof.

24. The process of claim 19 wherein an interactive website records and tracks location, survival rates, growth and carbon mass of trees grown from the tree seeds.

25. The process of claim 19 wherein the location, survival rates, growth and carbon mass of trees is verified by satellite imaging.
